SEQUENCE LISTING (2 sequences)

SEQ ID NO: 1 - protein, 406 aa, Artificial sequence
  Synthetic
ANAFLXXLRPGSLXRXCKXXQCSFXXARXIFKDAXRTKLFWISYSDGDQCASSPCQNGGSCKDQLQSYICFCLPAFEGRNCETHKDDQLICVNENGGCEQYCSDHTGTKRSCRCHEGYSLLADGVSCTPTVEYPCGKIPILEKRNASLPQTGIVGGKVCPKGECPWQVLLLVNGAQLCGGTLINTIWVVSAAHCFDKIKNWRNLIAVLGEHDLSEHDGDEQSRRVAQVIIPSTYVPGTTNHDIALLRLHQPVVLTDHVVPLCLPERTFSERTLAFVRFSLVSGWGQLLDRGATALELMVLNVPRLMTQDCLQQSRKVGDSPNITEYMFCAGYSDGSKDSCKGDSGGPHATHYRGTWYLTGIVSWGQGCATVGHFGVYTRVSQYIEWLQKLMRSEPRPGVLLRAPFP

SEQ ID NO: 2 - protein, 406 aa, Artificial sequence
  Synthetic
ANAFLXXLRPGSLXRXCKXXQCSFXXARXIFKDAXRTKLFWISYSDGDQCASSPCQNGGSCKDQLQSYICFCLPAFEGRNCETHKDDQLICVNENGGCEQYCSDHTGTKRSCRCHEGYSLLADGVSCTPTVEYPCGKIPILEKRNASNPQYNIVGGKVCPKGECPWQVLLLVNGAQLCGGTLINTIWVVSAAHCFDKIKNWRNLIAVLGEHDLSEHDGDEQSRRVAQVIIPSTYVPGTTNHDIALLRLHQPVVLTDHVVPLCLPERTFSERTLAFVRFSLVSGWGQLLDRGATALELMVLNVPRLMTQDCLQQSRKVGDSPNITEYMFCAGYSDGSKDSCKGDSGGPHATHYRGTWYLTGIVSWGQGCATVGHFGVYTRVSQYIEWLQKLMRSEPRPGVLLRAPFP

(19) United States
(12) Patent Application Publication
(10) Pub. No.: US 2009/0176967 A1
(43) Pub. Date: Jul. 9, 2009

(54) Conjugation of FVII

(75) Inventor: Henning Ralf Stennicke, Kokkedal (DK)

Correspondence Address: NOVO NORDISK, INC.;INTELLECTUAL PROPERTY DEPARTMENT, 100 COLLEGE ROAD WEST, PRINCETON, NJ, 08540, US

(73) Assignee: Novo Nordisk HealthCare A/G, Zurich (CH)

(21) Appl. No.: 11/659,153

(22) PCT Filed: Aug. 2, 2005
(86) PCT No.: PCT/EP2005/053756

§ 371 Date: Oct. 23, 2008

(30) Foreign Application Priority Data
Aug. 2, 2004 (DK) PA 2004 01175

(51) Int. Cl. C07K 14/745; C12P 21/06; C07K 5/10; C07K 7/06; C12N 15/12; C12N 15/81; C12N 1/19

(52) U.S. Cl. 530/330; 435/68.1; 530/381; 536/23.5; 435/320.1; 435/254.2

(57) ABSTRACT

New FVII polypeptides and FVIIa derivatives, uses of such peptides, and methods of producing these polypeptides and derivatives, are provided.

### FIELD OF THE INVENTION

The present invention relates to FVIIa, or variants thereof, derivatised in the C-terminus of the light chain as well as methods for achieving said derivatisation. The modification introduces moieties with protracting properties or functionalities which allows for further modification.

### BACKGROUND OF THE INVENTION

It is well-known to modify the properties and characteristics of peptides by conjugating groups to the peptide which changes the properties of the peptide. Such conjugation generally requires some functional group in the peptide to react with another functional group in a conjugating group. Typically, amino groups, such as the N-terminal amino group or the amino group in lysines, have been used in combination with a suitable acylating reagent. It is often desired or even required to be able to control the conjugation reaction, i.e. to control where the conjugating compounds are attached and to control how many conjugating groups are attached. This is often referred to as selectivity. The present invention provides a method for conjugating FVIIa specifically in the C-terminal.

### DESCRIPTION OF THE DRAWINGS

FIG. 1 describes the FVII polypeptide amino acid sequence for cleavage with sortase A (SEQ ID NO. 1).

FIG. 2 describes the FVII polypeptide amino acid sequence for cleavage with sortase B (SEQ ID NO. 2).

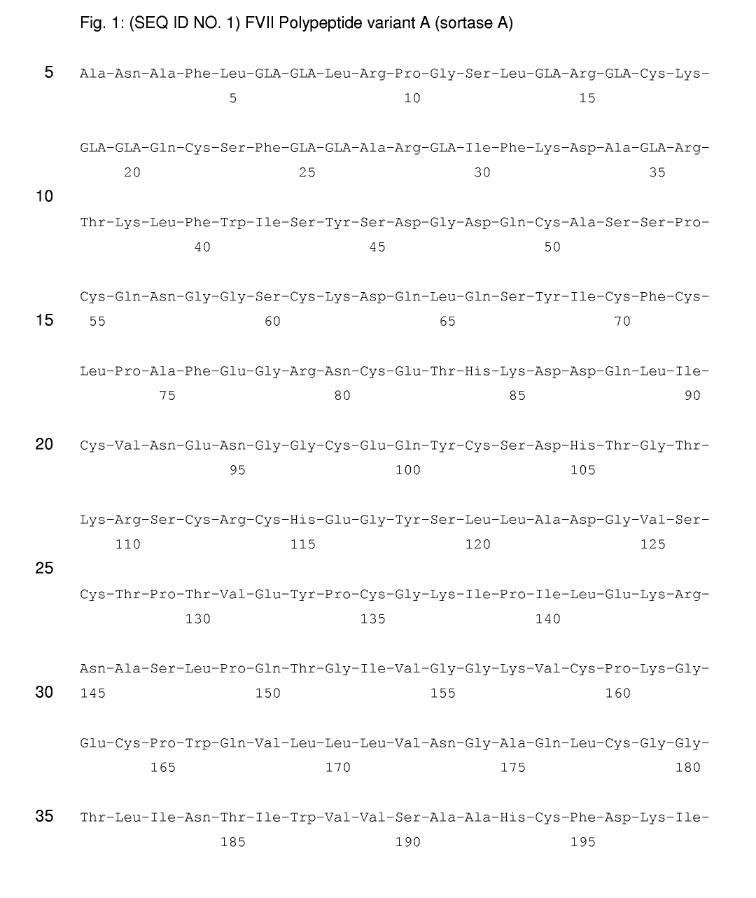

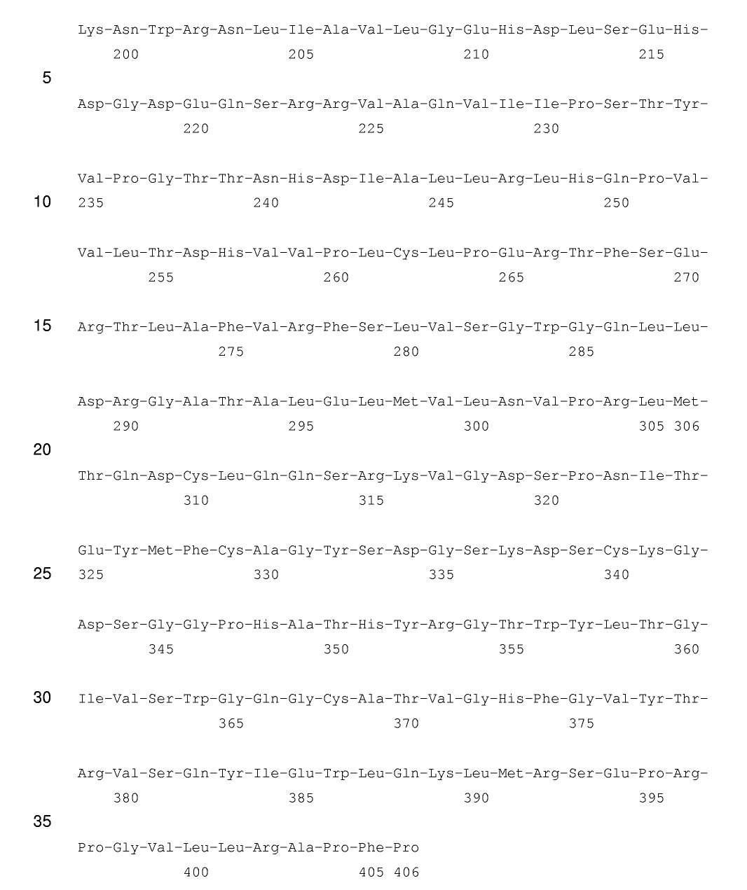

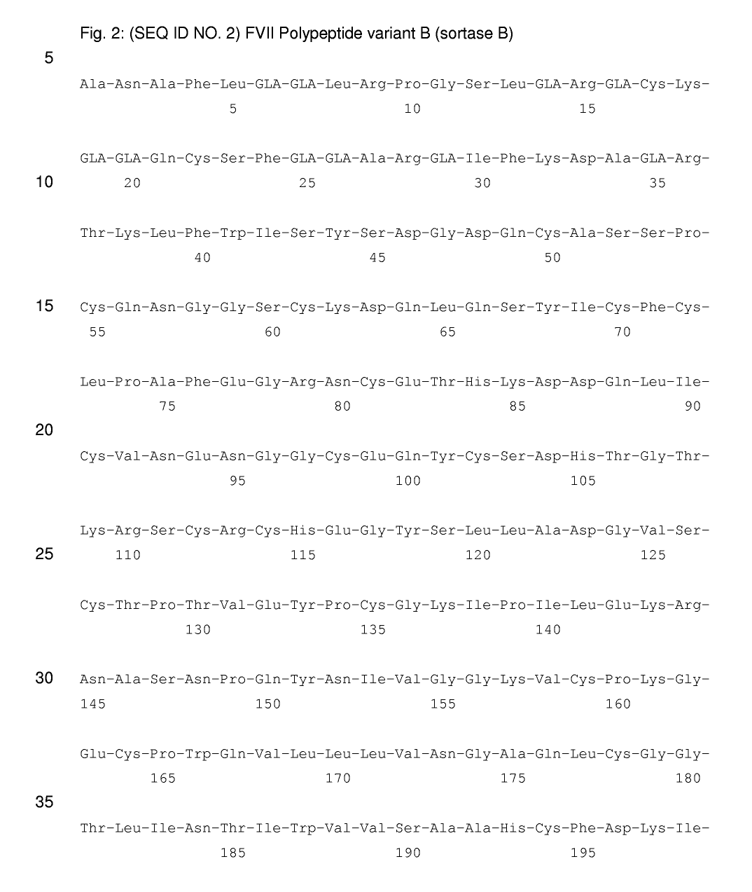

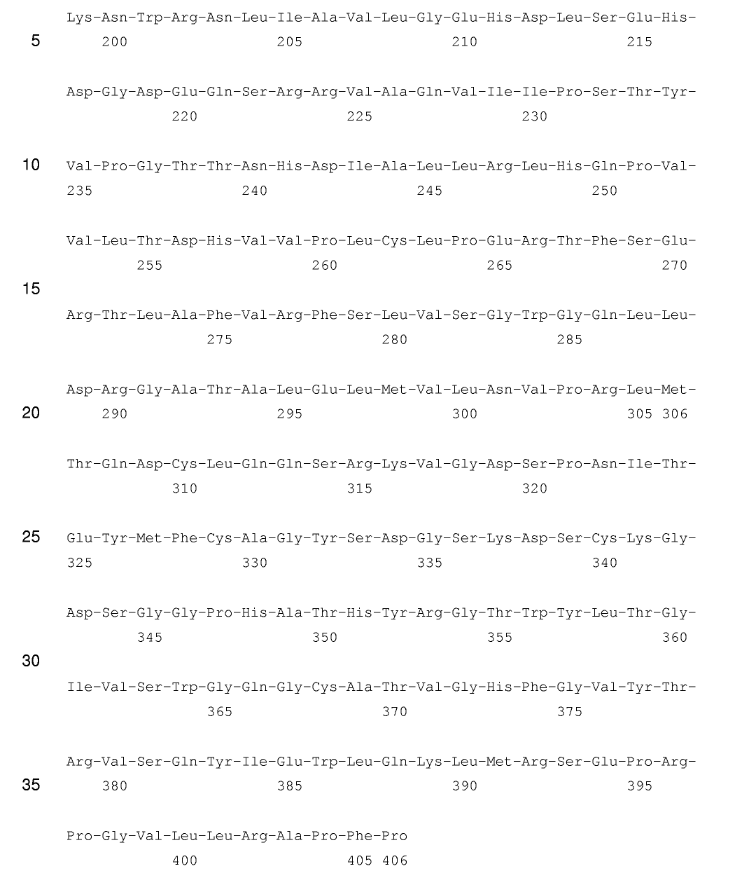

### SUMMARY OF THE INVENTION

The invention provides novel FVII polypeptides containing sites of cleavage by selected enzymes. The invention also provides DNA encoding such novel amino acid sequence variants and methods for expressing such peptides.

The invention provides a method of obtaining a derivate of FVIIa, P′—R—X, comprising the step of cleaving FVII or a FVII variant enzymatically in the presence of R′—X to conjugate —R—X at the enzymatically generated C-terminal of FVIIa:

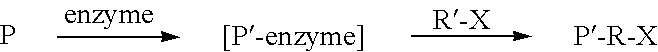

wherein P represents FVII or a FVII variant, P′ represents the product of the cleavage, R′—X represents the compound reacting with P′, X represents the group to be conjugated to P′ or X represents a functional group. R′ represents R comprising a nucleophilic group (—NH₂, —OH, or —SH), which in P′—R—X will be integrated into R as a linker part (—NH—, —O— or —S—).

The invention provides in an aspect of the above a method wherein the obtained product P′—R—X wherein X represents a functional group, is further reacted with a compound of the general formula Y-E-Z to obtain a product

P′—R-A-E-Z

wherein R represents a linker or a bond;
wherein P′ represents the product of the enzymatically cleavage of FVII;

X represents a radical comprising a functional group capable of reacting with Y;

Y represents a radical comprising one or more functional groups which groups are capable of reacting with X;

E represents a linker or a bond;

A represents the moiety formed by the reaction between the functional groups comprised in X and Y; and

Z is the moiety to be conjugated to the peptide.

### DEFINITIONS

In the present context, the term “oxime bond” is intended to indicate a moiety of the formula —C═N—O—.

In the present context, the term “hydrazone bond” is intended to indicate a moiety of the formula —C═N—N—.

In the present context, the term “phenylhydrazine bond” is intended to indicate a moiety of the formula

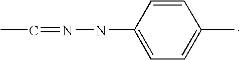

In the present context, the term “semicarbazone bond” is intended to indicate a moiety of the formula —C═N—N—C(O)—N—.

The term “alkane” is intended to indicate a saturated, linear, branched and/or cyclic hydrocarbon. Unless specified with another number of carbon atoms, the term is intended to indicate hydrocarbons with from 1 to 30 (both included) carbon atoms, such as 1 to 20 (both included), such as from 1 to 10 (both included), e.g. from 1 to 5 (both included).

The term “alkene” is intended to indicate linear, branched and/or cyclic hydrocarbons comprising at least one carbon-carbon double bond. Unless specified with another number of carbon atoms, the term is intended to indicate hydrocarbons with from 2 to 30 (both included) carbon atoms, such as 2 to 20 (both included), such as from 2 to 10 (both included), e.g. from 2 to 5 (both included).

The term “alkyne” is intended to indicate linear, branched and/or cyclic hydrocarbons comprising at least one carbon-carbon triple bond, and it may optionally comprise one or more carbon-carbon double bonds. Unless specified with another number of carbon atoms, the term is intended to indicate hydrocarbons with from 2 to 30 (both included) carbon atoms, such as from 2 to 20 (both included), such as from 2 to 10 (both included), e.g. from 2 to 5 (both included).

The term “homocyclic aromatic compound” is intended to indicate aromatic hydrocarbons, such as benzene and naphthalene.

The term “heterocyclic compound” is intended to indicate a cyclic compound comprising 5, 6 or 7 ring atoms from which 1, 2, 3 or 4 are hetero atoms selected from N, O and/or S. Examples of heterocyclic compounds include the aromatic heterocycles such as thiophene, furan, pyran, pyrrole, imidazole, pyrazole, isothiazole, isoxazole, pyridine, pyrazine, pyrimidine, pyridazine, as well as their partly or fully hydrogenated equivalents, such as piperidine, pyrazolidine, pyrrolidine, pyrroline, imidazolidine, imidazoline, piperazine and morpholine.

The terms “hetero alkane”, “hetero alkene” and “hetero alkyne” is intended to indicate alkanes, alkenes and alkynes as defined above, in which one or more hetero atom or group have been inserted into the structure of said moieties. Examples of hetero groups and atoms include —O—, —S—, —S(O)—, —S(O)₂—, —C(O)—, —C(S)— and —N(R*)-, wherein R* represents hydrogen or C₁-C₆-alkyl. Nonlimiting examples of heteroalkanes include:

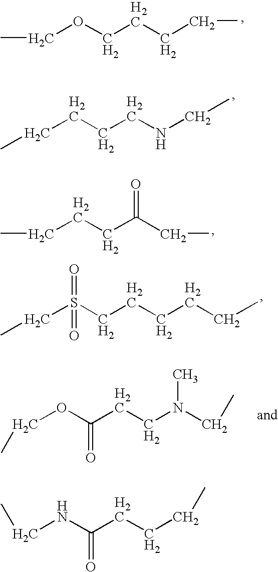

The term “radical” or “biradical” is intended to indicate a compound from which one or two, respectively, hydrogen atoms have been removed. When specifically stated, a radical may also indicate the moiety formed by the formal removal of a larger group of atoms, e.g. hydroxyl, from a compound.

The term “halogen” is intended to indicate F, Cl, Br and I.

The term “PEG” is intended to indicate polyethylene glycol of a molecular weight between 500 and 150,000 Da, including analogues thereof, wherein for instance the terminal OH-group has been replaced by a methoxy group (referred to as mPEG).

In the present context, the term “aryl” is intended to indicate a carbocyclic aromatic ring radical or a fused aromatic ring system radical wherein at least one of the rings are aromatic. Typical aryl groups include phenyl, biphenylyl, naphthyl, and the like.

The term “heteroaryl”, as used herein, alone or in combination, refers to an aromatic ring radical with for instance 5 to 7 member atoms, or to a fused aromatic ring system radical with for instance from 7 to 18 member atoms, wherein at least on ring is aromatic and containing one or more heteroatoms as ring atoms selected from nitrogen, oxygen, or sulfur heteroatoms, wherein N-oxides and sulfur monoxides and sulfur dioxides are permissible heteroaromatic substitutions. Examples include furanyl, thienyl, thiophenyl, pyrrolyl, imidazolyl, pyrazolyl, triazolyl, tetrazolyl, thiazolyl, oxazolyl, isoxazolyl, oxadiazolyl, thiadiazolyl, isothiazolyl, pyridinyl, pyridazinyl, pyrazinyl, pyrimidinyl, quinolinyl, isoquinolinyl, benzofuranyl, benzothiophenyl, indolyl, and indazolyl, and the like.

The term “conjugate” as a noun is intended to indicate a modified peptide, i.e. a peptide with a moiety bonded to it to modify the properties of said peptide. As a verb, the term is intended to indicate the process of bonding a moiety to a peptide to modify the properties of said peptide.

As used herein, the term “prodrug” indicates biohydrolyzable amides and biohydrolyzable esters and also encompasses a) compounds in which the biohydrolyzable functionality in such a prodrug is encompassed in the compound according to the present invention, and b) compounds which may be oxidized or reduced biologically at a given functional group to yield drug substances according to the present invention. Examples of these functional groups include 1,4-dihydropyridine, N-alkylcarbonyl-1,4-dihydropyridine, 1,4-cyclohexadiene, tert-butyl, and the like.

As used herein, the term “biohydrolyzable ester” is an ester of a drug substance (in casu, a compound according to the invention) which either a) does not interfere with the biological activity of the parent substance but confers on that substance advantageous properties in vivo such as duration of action, onset of action, and the like, or b) is biologically inactive but is readily converted in vivo by the subject to the biologically active principle. The advantage is, for example increased solubility or that the biohydrolyzable ester is orally absorbed from the gut and is transformed to a compound according to the present invention in plasma. Many examples of such are known in the art and include by way of example lower alkyl esters (e.g., C₁-C₄), lower acyloxyalkyl esters, lower alkoxyacyloxyalkyl esters, alkoxyacyloxy esters, alkyl acylamino alkyl esters, and choline esters.

As used herein, the term “biohydrolyzable amide” is an amide of a drug substance (in casu, a compound according to the present invention) which either a) does not interfere with the biological activity of the parent substance but confers on that substance advantageous properties in vivo such as duration of action, onset of action, and the like, or b) is biologically inactive but is readily converted in vivo by the subject to the biologically active principle. The advantage is, for example increased solubility or that the biohydrolyzable amide is orally absorbed from the gut and is transformed to a compound according to the present invention in plasma. Many examples of such are known in the art and include by way of example lower alkyl amides, α-amino acid amides, alkoxyacyl amides, and alkylaminoalkylcarbonyl amides.

In the present context, the term “pharmaceutically acceptable salt” is intended to indicate salts which are not harmful to the patient. Such salts include pharmaceutically acceptable acid addition salts, pharmaceutically acceptable metal salts, ammonium and alkylated ammonium salts. Acid addition salts include salts of inorganic acids as well as organic acids. Representative examples of suitable inorganic acids include hydrochloric, hydrobromic, hydroiodic, phosphoric, sulfuric, nitric acids and the like. Representative examples of suitable organic acids include formic, acetic, trichloroacetic, trifluoroacetic, propionic, benzoic, cinnamic, citric, fumaric, glycolic, lactic, maleic, malic, malonic, mandelic, oxalic, picric, pyruvic, salicylic, succinic, methanesulfonic, ethanesulfonic, tartaric, ascorbic, pamoic, bismethylene salicylic, ethanedisulfonic, gluconic, citraconic, aspartic, stearic, palmitic, EDTA, glycolic, p-aminobenzoic, glutamic, benzenesulfonic, p-toluenesulfonic acids and the like. Further examples of pharmaceutically acceptable inorganic or organic acid addition salts include the pharmaceutically acceptable salts listed in J. Pharm. Sci. 1977, 66, 2, which is incorporated herein by reference. Examples of metal salts include lithium, sodium, potassium, magnesium salts and the like. Examples of ammonium and alkylated ammonium salts include ammonium, methylammonium, dimethylammonium, trimethylammonium, ethylammonium, hydroxyethylammonium, diethylammonium, butylammonium, tetramethylammonium salts and the like.

A “therapeutically effective amount” of a compound as used herein means an amount sufficient to cure, alleviate or partially arrest the clinical manifestations of a given disease and its complications. An amount adequate to accomplish this is defined as “therapeutically effective amount”. Effective amounts for each purpose will depend on the severity of the disease or injury as well as the weight and general state of the subject. It will be understood that determining an appropriate dosage may be achieved using routine experimentation, by constructing a matrix of values and testing different points in the matrix, which is all within the ordinary skills of a trained physician or veterinary.

The term “treatment” and “treating” as used herein means the management and care of a patient for the purpose of combating a condition, such as a disease or a disorder. The term is intended to include the full spectrum of treatments for a given condition from which the patient is suffering, such as administration of the active compound to alleviate the symptoms or complications, to delay the progression of the disease, disorder or condition, to alleviate or relief the symptoms and complications, and/or to cure or eliminate the disease, disorder or condition as well as to prevent the condition, wherein prevention is to be understood as the management and care of a patient for the purpose of combating the disease, condition, or disorder and includes the administration of the active compounds to prevent the onset of the symptoms or complications. The patient to be treated is preferably a mammal, in particular a human being, but it may also include animals, such as dogs, cats, cows, sheep and pigs.

The term “functional in vivo half-life” is used in its normal meaning, i.e., the time at which 50% of the biological activity of the polypeptide or conjugate is still present in the body/target organ, or the time at which the activity of the polypeptide or conjugate is 50% of its initial value. As an alternative to determining functional in vivo half-life, “serum half-life” may be determined, i.e., the time at which 50% of the polypeptide or conjugate molecules circulate in the plasma or bloodstream prior to being cleared. Determination of serum-half-life is often more simple than determining functional half-life and the magnitude of serum-half-life is usually a good indication of the magnitude of functional in vivo half-life. Alternative terms to serum half-life include plasma half-life, circulating half-life, circulatory half-life, serum clearance, plasma clearance, and clearance half-life. The functionality to be retained is normally selected from procoagulant, proteolytic, co-factor binding, receptor binding activity, or other type of biological activity associated with the particular protein.

The term “increased” as used about the functional in vivo half-life or plasma half-life is used to indicate that the relevant half-life of the polypeptide or conjugate is statistically significantly increased relative to that of a reference molecule, such as non-conjugated glycoprotein as determined under comparable conditions. For instance the relevant half-life may be increased by at least about 25%, such as by at least about 50%, e.g., by at least about 100%, 150%, 200%, 250%, or 500%.

The term “polymeric molecule”, or “polymeric group” or “polymeric moiety” or “polymer molecule”, encompasses molecules formed by covalent linkage of two or more monomers wherein none of the monomers is an amino acid residue. Preferred polymers are polymer molecules selected from the group consisting of dendrimers as disclosed in Danish Patent Application No. PA 2003 01145, polyalkylene oxide (PAO), including polyalkylene glycol (PAG), such as polyethylene glycol (PEG) and polypropylene glycol (PPG), branched PEGs, polyvinyl alcohol (PVA), polycarboxylate, poly-vinylpyrrolidone, polyethylene-co-maleic acid anhydride, polystyrene-co-maleic acid anhydride, and dextran, including carboxymethyl-dextran, PEG being particularly preferred.

“Immunogenicity” of a preparation refers to the ability of the preparation, when administered to a human, to elicit a deleterious immune response, whether humoral, cellular, or both. In any human sub-population, there may exist individuals who exhibit sensitivity to particular administered proteins. Immunogenicity may be measured by quantifying the presence of anti-protein antibodies and/or protein responsive T-cells in a sensitive individual, using conventional methods known in the art. In some embodiments, the preparations of the present invention exhibit a decrease in immunogenicity in a sensitive individual of at least about 10%, preferably at least about 25%, more preferably at least about 40% and most preferably at least about 50%, relative to the immunogenicity for that individual of a reference preparation.

The term “protractor group” or, interchangeably, “protractor moiety” is intended to include groups that, when covalently attached to the protein, protract the serum half-life of the conjugated protein compared to the non-conjugated protein. The prolonged activity is achieved by preventing or decreasing clearance (specific or non-specific) of the particular glycoprotein. The conjugated glycoprotein should substantially preserve its biological activity. Non-limiting examples include polymeric groups such as, e.g, dendrimers as disclosed in Danish Patent Application No. PA 2003 01145, polyalkylene oxide (PAO), polyalkylene glycol (PAG), polyethylene glycol (PEG), polypropylene glycol (PPG), branched PEGs, polyvinyl alcohol (PVA), polycarboxylate, poly-vinylpyrrolidone, polyethylene-co-maleic acid anhydride, polystyrene-co-maleic acid anhydride, dextran, carboxymethyl-dextran; serum protein binding-ligands, such as compounds which bind to albumin, like fatty acids, C₅-C₂₄ fatty acid, aliphatic diacid (e.g. C₅-C₂₄). Albumin binders are described in Danish patent applications PA 2004 01083, PA 2003 01788 and PA 2003 01366. Albumin binders are also compounds of the following formula:

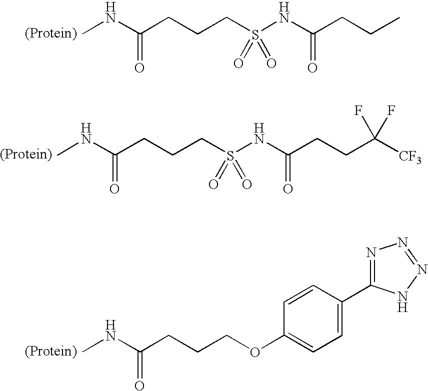

Other examples of protractor groups includes small organic molecules containing moieties that under physiological conditions alters charge properties, such as carboxylic acids or amines, or neutral substituents that prevent glycan specific recognition such as smaller alkyl substituents (e.g., C₁-C₅ alkyl).

In the present context, the words “peptide” and “polypeptide” and “protein” are used interchangeably and are intended to indicate the same.

### DESCRIPTION OF THE INVENTION

The present invention provides a method for incorporating functional groups into FVIIa at a predetermined site.

The present invention provides a method by which FVII may be conjugated with a high degree of selectivity at the C-terminus of the FVIIa light chain. One method exploit the ability of FVIIa to convert FVII into FVIIa in an auto-catalytic process, by specifically cleaving at the C-terminus of the FVII light chain. In this cleavage reaction other nucleophiles than water, e.g., primary amines, may be utilized in the de-acylation of FVIIa thus, incorporating a functional group into the C-terminal end of the light chain of FVIIa, where said functional group is subsequently used as a conjugation point. The above reaction requires two very specific abilities of the cleaving enzyme, i.e., the ability to specifically cleave between the light and heavy chain of FVII and the ability to utilize other nucleophiles than water to catalyze de-acylation, many other enzymes may be used in place of FVIIa to catalyze/initiate the reaction. These enzymes includes FXa, FSAP, Hepsin etc. In an alternative scenario enzymes which depend on amine nucleophiles, e.g. sortases, may be utilized in combination with FVII variants containing a genetically introduced recognition site.

Compared to other conjugation methods which take advantage of functional groups already present in the peptide, e.g. terminal amino groups or ε-amino groups of lysines, the method of the present invention offers the advantage of improved selectivity. The incorporation of one or more functional groups not accessible in the peptide ensures that the conjugation takes place at only specified loci. Furthermore, the invention provides derivatisation at a loci in FVII, which is a naturally cleavage point to give a fully functional peptide.

Schematically represented the reaction proceeds as follows:

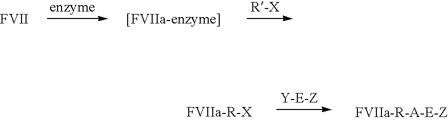

FVII or a variant of FVII is specifically cleaved between R¹⁵² and Ile¹⁵³ and thereby forming the active form, which is FVIIa. R′—X represents the compound reacting with FVIIa. X represents the group to be conjugated to FVIIa or X represents a functional group. R′ represents R comprising a nucleophilic group (—NH₂, —OH, or —SH), which in FVIIa-R—X will be integrated into R as a linker part (—NH—, —O— or —S—).

In principle any serine/thiol protease capable of cleaving the natural activation site (PQGR¹⁵²—I¹⁵³VGG) while adding a nucleophile other than HO to R¹⁵² may be used. Alternatively, a sequence suitable for another protease or protein transferase, e.g. sortase (ref. Kruger et al. Biochemistry. 2004 Feb. 17; 43(6):1541-51.), may be introduced prior to the sequence -I¹⁵³VGG to yield an optimal site for another enzyme.

Theoretically, trans-acylation may be mediated by all proteolytic enzymes via either a kinetically controlled or an equilibrium controlled mechanism for enzymes with or without a covalent intermediate, respectively. For FVIIa we have focused on enzymes which depend in the existence of a covalent ester/thioester intermediate, i.e., enzymes in which the catalytic apparatus includes a serine or a cysteine. The mechanism of action then becomes a two step reaction in which the substrate is bound and the covalent intermediate formed (acylation) followed by the nucleophilic attack on the intermediate by a nucleophile other than water (transacylation). Naturally, as most of the enzymes are designed to hydrolyze the peptide bond the transacylation occurs in competition with the hydrolysis or deacylation reaction.

The number and type of cleavage proteins is further expanded by insertion of a specific cleavage site into FVII. In practicing the present invention, any proteolytic enzyme may be used, as long as the amino acid sequence IVGG is tolerated on the aminoterminal side of the cleavage site and the enzyme is sufficiently selective to provide only a single cleavage in the entire polypeptide, that cleavage occurring at the activation site of FVIIa. In one series of embodiments, the cleavage site may be modified to LPXTG-IVGG or NPXTN-IVGG to act as substrates for sortase A or B, respectively (Kruger et al. Biochemistry. 2004; 43:1541-51)

Relevante enzymes includes sortase A from Staphylococcus aureus, Bacillus anthracis, Bacillus cereus, Bacillus halodurans, Clostridium acetobutylicum, Clostridium perfringens, Clostridium tetani, Enterococcus faecalis, Lactobacillus plantarum, Lactococcus lactis, Listeria innocua, Listeria monocytogenes, Stephylococcus epidermis, Streptococcus agalactiae, Streptococcus gordonii, Streptococcus mutans, Streptococcus phenumoniae, Streptococcus pyogenes, Streptococcus suis.

Relevante enzymes include sortase B from Staphylococcus aureus, Bacillus anthracis, Bacillus cereus, Bacillus halodurans, Clostridium perfringens, Listeria innocua, Listeria monocytogenes.

In an embodiment of the invention sortase A and B from Staphylococcus aureus is use.

In the case where the natural cleavage site is used the obvious group of protease for modification includes enzymes which previously have been demonstrated to be able to activate FVII, i.e., FVIIa it self as well as FIXa (JBC 272, 17467-72), FXa, FSAP (Romisch, Biol. Chem. 2002, 383:1119-24), Hepsin (Kazama et al. J Biol. Chem. 1995, 270:66-72.) and matriptase (H. R. Stennicke—unpublished).

In either of the above cases an intermediate is formed which is an activated derivative of FVIIa modified at the C-terminus of the light chain by a protractive or a functional group which may be further modified. In the case of a functional group the FVIIa derivative is subsequently reacted with another compound comprising one or more functional group groups which reacts specifically with the activated FVIIa derivative.

In principle any primary amine should be able to act as R—X for the de-acylation reaction, however, factors like pKₐ, steric hindrance, affinity and solubility will affect the potency and efficiency of the nucleophile.

Many nucleophilic compounds are known which could be incorporated into peptides according to the methods of the present invention, and α-amino acids is one such type of nucleophilic compounds. For the purpose of the present invention, it is, however, preferred to select the nucleophilic compound so that the product of the reaction is not itself a substrate for the enzyme applied.

Whether or not a compound is a substrate for a given enzyme in principle depends on the conditions, e.g. the time under which the reaction takes place. Given sufficient time, many compounds are, in fact, substrates for an enzyme although they are not under normal conditions regarded as such. When it is stated above that the product itself should not be a substrate of the enzyme it is intended to indicate that the product itself is not a substrate for the enzyme to an extent where the following reactions in the method of the present invention is disturbed. If the product is, in fact, a substrate for the enzyme, the enzyme may be removed or inactivated, e.g. by enzyme inhibitors, following the reaction.

The reaction of the peptide and the nucleophile affords a transacylated peptide wherein the C-terminal amino acid residue or peptide (in the case of FVII the peptide is the heavy chain) has been exchanged with the nucleophilic compound, which comprises one or more functional groups which are not accessible in the peptide to be conjugated. The overall result of this reaction (or this series of reactions) is an incorporation of one or more functional groups into the peptide which are present at only one locus in the peptide. A subsequent reaction (or series of reactions) with a compound comprising the moiety to be conjugated to the peptide and one or more functional groups which only react with the functional groups added to the peptide in the transacylation reaction effects a selective conjugation of the peptide to be conjugated.

In an aspect of the invention R′—X is an α-amino acid derivative

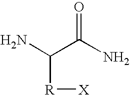

in the presence of the chosen enzyme for cleavage of FVII into FVIIa forms a compound of the formula

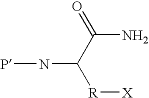

wherein P′ represent the FVIIa.

In another embodiment of the invention the R—X is a peptide modified in the C-terminal and optionally in one or more of the amino acids. The unmodified N-terminal acts as a nucleophile, attaching to the peptide P′ a peptide sequence having a C-terminal amide. One of the amino acids in the sequence thus carries the modification X to which further attachments can be made. The peptide can in principle be any length and can carry one or more modified amino acids.

The compound to be reacted with the FVIIa or the intermediate P′—R—X, comprises a linker, R and E, each comprising a nucleophilic group, respectively. These linkers, which are independent of each other, may be absent or selected from amongst alkane, alkene or alkyne diradicals and hetero alkane, hetero alkene and hetero alkyne diradicals, wherein one or more optionally substituted aromatic homocyclic biradical or biradical of a heterocyclic compound, e.g. phenylene or piperidine biradical may be inserted into the aforementioned biradicals. It is to be understood that said linkers may also comprise substitutions by groups selected from amongst hydroxyl, halogen, nitro, cyano, carboxyl, aryl, alkyl and heteroaryl. In an aspect of the invention the linker includes also amino acids forming small peptides. The functionalities X or Y as described above may be either internally in the amino acid sequence or in either of the terminals. The X's and Y's are optionally inserted as amino acid derivatives carrying the desired functionality.

In an aspect of the invention the R′—X is a small sequences of peptide amides wherein one of the amino acids is derivatised to contain the group X as described. In an aspect of the invention the peptide is 1-20 amino acids.

A general example of R′—X is (AA)ₐ-(A^{x})-(AA)_{b}-NH₂

wherein AA represents any amino acid, A^{x} represent the derivative carrying X, a and b denotes any number including 0.

Y-E-Z represents the moiety introducing the group Z. Y is selected to react with X in P′—R—X forming the group A in P′—R-A-E-Z.

The moiety, A, formed in the reaction between the functional groups of X and Y may in principle be of any kind depending on what properties of the final conjugated peptide is desired. In some situation it may be desirable to have a labile bond which can be cleaved at some later stage, e.g. by some enzymatic action or by photolysis. In other situations, it may be desirable to have a stable bond, so that a stable conjugated peptide is obtained. Particular mentioning is made of the type of moieties formed by reactions between amine derivatives and carbonyl groups, such as oxime, hydrazone, phenylhydrazine and semicarbazone moieties.

In one embodiment the functional groups of X and Y are selected from amongst carbonyl groups, such as keto and aldehyde groups, and amino derivatives, such as

hydrazine derivatives —NH—NH₂,
hydrazine carboxylate derivatives —O—C(O)—NH—NH₂,
semicarbazide derivatives —NH—C(O)—NH—NH₂,
thiosemicarbazide derivatives —NH—C(S)—NH—NH₂,
carbonic acid dihydrazide derivatives —NHC(O)—NH—NH—C(O)—NH—NH₂,
carbazide derivatives —NH—NH—C(O)—NH—NH₂,
thiocarbazide derivatives —NH—NH—C(S)—NH—NH₂,
aryl hydrazine derivatives —NH—C(O)—C₆H₄—NH—NH₂, and
hydrazide derivatives —C(O)—NH—NH₂;
oxylamine derivatives, such as —O—NH₂, —C(O)—O—NH₂, —NH—C(O)—O—NH₂ and —NH—C(S)—O—NH₂.

It is to be understood, that if the functional group comprised in X is a carbonyl group, then the functional group comprised in Y is an amine derivative, and vice versa.

In an aspect of the invention Y is a group of the form of —O—NH₂,

Another example of a suitable pair of X and Y is azide derivatives (—N₃) and alkynes which react to form a triazole moiety.

Both E and R in the formula P′—R-A-E-Z represent bonds or linkers, and in the present context the term “linker” is intended to indicate a moiety functioning as a means to separate Y from Z and X from the peptide, respectively. One function of the linkers E and R may be to provide adequate flexibility in the linkage between the peptide and the conjugated moiety Z. Typical examples of E and R′ include straight, branched and/or cyclic C₁₋₁₀alkylene, C₂₋₁₀alkenylene, C₂₋₁₀alkynylene, C₁₋₂₂heteroalkylene, C₂₋₁₀heteroalkenylene, C₂₋₁₀heteroalkynylene, wherein one or more homocyclic aromatic compound biradical or heterocyclic compound biradical may be inserted. Particular examples of E and R include

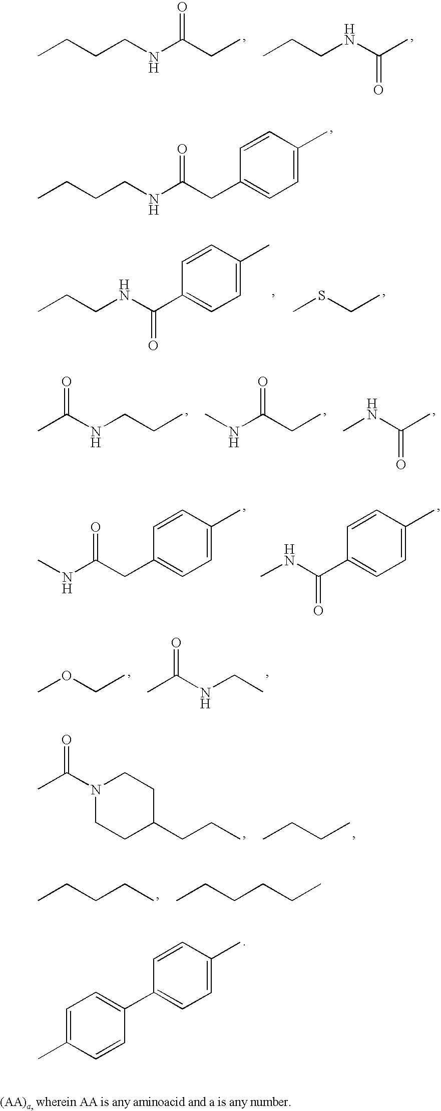

In an embodiment of the invention Z is a protractor group. In an embodiment of the invention Z is a PEG group. The PEG conjugated to a peptide according to the present invention may be of any molecular weight. In particular the molecular weight may be between 500 and 100,000 Da, such as between 500 and 60,000 Da, such as between 1000 and 40,000 Da, such as between 5,000 and 40,000 Da. In particular, PEG with molecular weights of 10,000 Da, 20,000 Da or 40,000 KDa may be used in the present invention. In all cases the PEGs may be linear or branched.

Z may be branched so that Z comprises more than one label or radical. A branched PEG may for example include 2 PEG molecules of each 10,000 KDa or 20,000 KDa or combinations of two PEG molecules of different weight.

In one embodiment, Z comprises one or more moieties that are known to bind to plasma proteins, such as e.g. albumin. The ability of a compound to bind to albumin may be determined as described in J. Med. Chem., 43, 2000, 1986-1992, which is incorporated herein by reference. In the present context, a compound is defined as binding to albumin if the ratio of the Resonance Units (Ru) to molecular weight (MW) as measured in Da is above 0.05, such as above 0.10, such as above 0.12 or even above 0.15 as measured according to J. Med. Chem., 43, 2000, 1986-1992.

In another embodiment of the invention the albumin binding moiety is a peptide, such as a peptide comprising less than 40 amino acid residues. A number of small peptides which are albumin binding moieties are disclosed in J. Biol. Chem. 277, 38 (2002) 35035-35043, which is incorporated herein by reference.

Albumin binders are described in Danish patent applications PA 2004 01083, PA 2003 01788 and PA 2003 01366. Albumin binders (below represented with the protein and linker in brackets) are also compounds of the following formula:

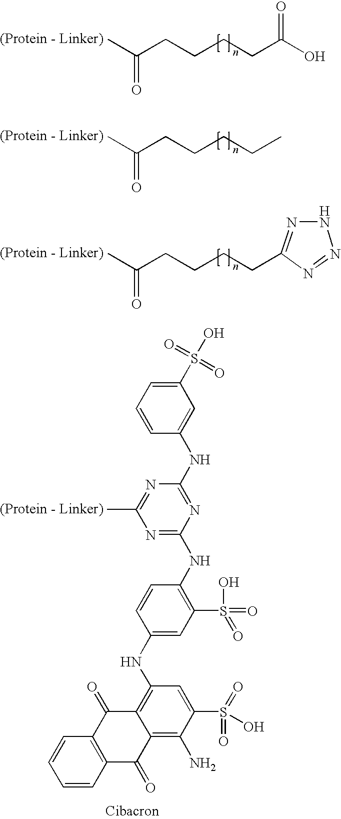

In an embodiment of the invention PEGylated human Factor VIIa is prepared according to the present invention.

Particular examples of Z include labels, such as fluorescent markers, such as fluorescein radical, rhodamine radical, Texas Red® radical and phycobili protein radical; enzyme substrates, such as p-nitrophenol acetate radical; and organic ligands in complex with radioactive isotopes, such as Cu-64, Ga67, Ga-68, Zr-89, Ru-97, Tc-99, Rh-105, Pd-109, In-111, I-123, I-125, I-131, Re-186, Re-188, Au-198, Pb-203, At-211, Pb-212 and Bi-212; organic moieties, such as PEG or mPEG radicals and amino derivatives thereof; straight, branched and/or cyclic C₁₋₂₂alkyl, C₂₋₂₂alkenyl, C₂₋₂₂alkynyl, C₁₋₂₂heteroalkyl, C₂₋₂₂heteroalkenyl, C₂₋₂₂heteroalkynyl, wherein one or more homocyclic aromatic compound biradical or heterocyclic compound biradical may be inserted, and wherein said C₁-C₂₂ or C₂-C₂₂ radicals may optionally be substituted with one or more substituents selected from hydroxyl, halogen, carboxyl, heteroaryl and aryl, wherein said aryl or heteroaryl may optionally be further substituted by one or more substituents selected from hydroxyl, halogen, and carboxyl; steroid radicals; lipid radicals; polysaccharide radicals, e.g. dextrans; polyamide radicals e.g. polyamino acid radicals; PVP radicals; PVA radicals; poly(1-3-dioxolane); poly(1,3,6-trioxane); ethylene/maleic anhydride polymer; Cibacron dye stuffs, such as Cibacron Blue 3GA, and polyamide chains of specified length, as disclosed in WO 00/12587, which is incorporated herein by reference.

Particular mentioning is made of C₁₀₋₂₀alkyl, such as C₁₅ and C₁₇, and benzophenone derivatives of the formula

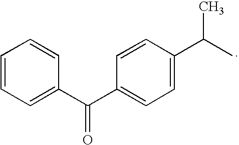

Particular examples of compounds of the formula Y-E-Z include

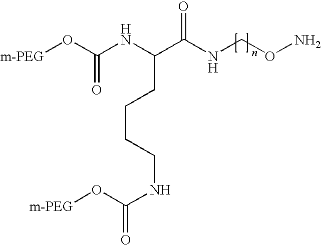

wherein n is 1, 2, 3, 4, 5 or 6 and mPEG has a molecular weight of 10 kDa, 20 kDa, 30 kDa or 40 kDa.

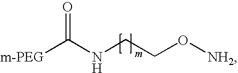

wherein m is 1, 2, 3, 4, 5 or 6 and mPEG has a molecular weight of 10 kDa, 20 kDa, 30 kDa or 40 kDa.

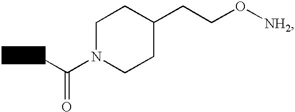

wherein mPEG has a molecular weight of 10 kDa, 20 kDa, 30 kDa or 40 kDa,

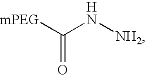

wherein mPEG has a molecular weight of 10 kDa, 20 kDa, 30 kDa or 40 kDa,

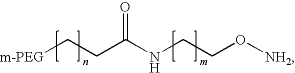

wherein n is 0, 1, 2, 3, 4, 5 or 6 and m is 1, 2, 3, 4, 5 or 6 and mPEG has a molecular weight of 10 kDa, 20 kDa, 30 kDa or 40 kDa,

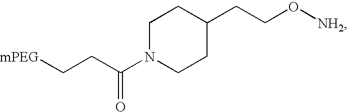

wherein mPEG has a molecular weight of 10 kDa, 20 kDa, 30 kDa or 40 kDa,

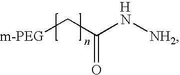

wherein n is 1, 2, 3, 4, 5 or 6 and mPEG has a molecular weight of 10 kDa, 20 kDa, 30 kDa or 40 kDa,

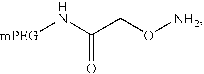

wherein mPEG has a molecular weight of 10 kDa, 20 kDa, 30 kDa or 40 kDa,

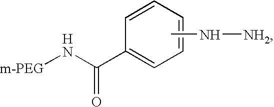

wherein mPEG has a molecular weight of 10 kDa, 20 kDa, 30 kDa or 40 kDa,

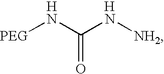

wherein mPEG has a molecular weight of 10 kDa, 20 kDa, 30 kDa or 40 kDa,

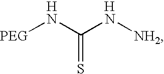

wherein mPEG has a molecular weight of 10 kDa, 20 kDa, 30 kDa or 40 kDa,

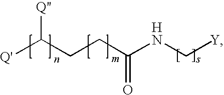

wherein Y is —O—NH₂, NH—NH₂,

n, m and s are independently selected from any number from 0 to 20, such as above 2;

Q′ and Q″ independently represents for example hydrogen, methyl, phenyl, biphenyl, phenoxyphenyl, phenylcarboxyphenyl.

At any suitable position in the alkyl chains in any of the formulas above a group of the formula —SO₂—, —C(O)NH—, —C(O)NHSO₂—, —SO₂-phenyl-, —C(O)NHSO₂-phenyl- may be inserted in either direction. Optionally the group C(O)NH in the above formula may be substituted by

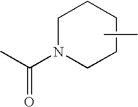

In an embodiment of the invention the introduction of the derivative Z is introduced in one step. The R—X then contains the derivatives to be introduced into FVIIa and could be described as R-A-E-Z. The nucleophile represents for example amino acids, which has been modified to carry the derivative. In principle any sequence of amino acids may be used. In an aspect of the invention nucleophiles such as G₍₁₋₅₎-PEG, G₍₁₋₅₎-lipid, G₍₁₋₄₎-NH—CH₂—CHO, G₍₁₋₄₎-NH—(CH₂)ₙ—O—NH₂, wherein n is ≧2, such as 2 etc. are used.

A need for modifying peptides may arise for any number of reasons, and this is also reflected in the kinds of compounds that may be conjugated to peptides according to the methods of the present invention. It may be desirable to conjugate peptides to alter the physico-chemical properties of the peptide, such as e.g. to increase (or to decrease) solubility to modify the bioavailability of therapeutic peptides. In another embodiment, it may be desirable to modify the clearance rate in the body by conjugating compounds to the peptide which binds to plasma proteins, such as e.g. albumin, or which increase the size of the peptide to prevent or delay discharge through the kidneys. In another embodiment, it may be desirable to conjugate a label to facilitate analysis of the peptide. Examples of such label include radioactive isotopes, fluorescent markers and enzyme substrates. In still another embodiment, a compound is conjugated to a peptide to facilitate isolation of the peptide. For example, a compound with a specific affinity to a particular column material may be conjugated to the peptide. It may also be desirable to modify the immunogenicity of a peptide, e.g. by conjugating a peptide so as to hide, mask or eclipse one or more immunogenic epitopes at the peptide. In an aspect of the invention the obtained peptides has improved biological half-life. In an aspect of the invention the peptides has improved activity as compared to the native peptide. In an aspect of the invention the obtained peptides has maintained it activity as compared to the native peptide.

In an embodiment of the present invention the functional in vivo half-life is increased by adding a polymeric molecule to the c-terminal of FVIIa. In an embodiment of the invention this is a protractor group. In an embodiment of the invention this is a PEG group. The PEG conjugated to a peptide according to the present invention may be of any molecular weight. In particular the molecular weight may be between 500 and 100,000 Da, such as between 500 and 60,000 Da, such as between 1000 and 40,000 Da, such as between 5,000 and 40,000 Da. In particular, PEG with molecular weights of 10,000 Da, 20,000 Da or 40,000 KDa may be used in the present invention. In all cases the PEGs may be linear or branched.

The term “Factor VII derivative” as used herein, is intended to designate wild-type Factor VII, variants of Factor VII exhibiting substantially the same or improved biological activity relative to wild-type Factor VII and Factor VII-related polypeptides, in which one or more of the amino acids of the parent peptide have been chemically modified, e.g. by alkylation, PEGylation, acylation, ester formation or amide formation or the like. This includes but are not limited to PEGylated human Factor VIIa, cysteine-PEGylated human Factor VIIa and variants thereof.

As used herein, “Factor VII-related polypeptides” encompasses polypeptides, including variants, in which the Factor VIIa biological activity has been substantially modified or reduced relative to the activity of wild-type Factor VIIa. These polypeptides include, without limitation, Factor VII or Factor VIIa into which specific amino acid sequence alterations have been introduced that modify or disrupt the bioactivity of the polypeptide.

Factor VII variants having substantially the same or improved biological activity relative to wild-type Factor VIIa encompass those that exhibit at least about 25%, preferably at least about 50%, more preferably at least about 75% and most preferably at least about 90% of the specific activity of Factor VIIa that has been produced in the same cell type, when tested in one or more of a clotting assay, proteolysis assay, or TF binding assay as described above. Factor VII variants having substantially reduced biological activity relative to wild-type Factor VIIa are those that exhibit less than about 25%, preferably less than about 10%, more preferably less than about 5% and most preferably less than about 1% of the specific activity of wild-type Factor VIIa that has been produced in the same cell type when tested in one or more of a clotting assay, proteolysis assay, or TF binding assay as described above. Factor VII variants having a substantially modified biological activity relative to wild-type Factor VII include, without limitation, Factor VII variants that exhibit TF-independent Factor X proteolytic activity and those that bind TF but do not cleave Factor X.

Variants of Factor VII, whether exhibiting substantially the same or better bioactivity than wild-type Factor VII, or, alternatively, exhibiting substantially modified or reduced bioactivity relative to wild-type Factor VII, include, without limitation, peptides having an amino acid sequence that differs from the sequence of wild-type Factor VII by insertion, deletion, or substitution of one or more amino acids.

The terms “variant” or “variants”, as used herein, is intended to designate Factor VII having the sequence of wild-type factor VII, wherein one or more amino acids of the parent protein have been substituted by another amino acid and/or wherein one or more amino acids of the parent protein have been deleted and/or wherein one or more amino acids have been inserted in protein and/or wherein one or more amino acids have been added to the parent protein. Such addition can take place either at the N-terminal end or at the C-terminal end of the parent protein or both. The “variant” or “variants” within this definition still have FVII activity in its activated form. In one embodiment a variant is 70% identical with the sequence of wild-type Factor VII. In one embodiment a variant is 80% identical with the sequence of wild-type factor VII. In another embodiment a variant is 90% identical with the sequence of wild-type factor VII. In a further embodiment a variant is 95% identical with the sequence of wild-type factor VII.

Non-limiting examples of Factor VII variants having substantially the same or increased proteolytic activity compared to recombinant wild type human Factor VIIa include S52A-FVIIa, S60A-FVIIa (Lino et al., Arch. Biochem. Biophys. 352: 182-192, 1998); FVIIa variants exhibiting increased proteolytic stability as disclosed in U.S. Pat. No. 5,580,560; Factor VIIa that has been proteolytically cleaved between residues 290 and 291 or between residues 315 and 316 (Mollerup et al., Biotechnol. Bioeng. 48:501-505, 1995); oxidized forms of Factor VIIa (Kornfelt et al., Arch. Biochem. Biophys. 363:43-54, 1999); FVII variants as disclosed in PCT/DK02/00189 (corresponding to WO 02/077218); and FVII variants exhibiting increased proteolytic stability as disclosed in WO 02/38162 (Scripps Research Institute); FVII variants having a modified Gla-domain and exhibiting an enhanced membrane binding as disclosed in WO 99/20767, U.S. Pat. No. 6,017,882 and U.S. Pat. No. 6,747,003, US patent application 20030100506 (University of Minnesota) and WO 00/66753, US patent applications US 20010018414, US 2004220106, and US 200131005, U.S. Pat. No. 6,762,286 and U.S. Pat. No. 6,693,075 (University of Minnesota); and FVII variants as disclosed in WO 01/58935, U.S. Pat. No. 6,806,063, US patent application 20030096338 (Maxygen ApS), WO 03/93465 (Maxygen ApS), WO 04/029091 (Maxygen ApS), WO 04/083361 (Maxygen ApS), and WO 04/111242 (Maxygen ApS), as well as in WO 04/108763 (Canadian Blood Services).

Non-limiting examples of FVII variants having increased biological activity compared to wild-type FVIIa include FVII variants as disclosed in WO 01/83725, WO 02/22776, WO 02/077218, PCT/DK02/00635 (corresponding to WO 03/027147), Danish patent application PA 2002 01423 (corresponding to WO 04/029090), Danish patent application PA 2001 01627 (corresponding to WO 03/027147); WO 02/38162 (Scripps Research Institute); and FVIIa variants with enhanced activity as disclosed in JP 2001061479 (Chemo-Sero-Therapeutic Res Inst.), all of which are incorporated herein by reference.

Examples of variants of factor VII include, without limitation, L305V-FVII, L305V/M306D/D309S-FVII, L3051-FVII, L305T-FVII, F374P-FVII, V158T/M298Q-FVII, V158D/E296V/M298Q-FVII, K337A-FVII, M298Q-FVII, V158D/M298Q-FVII, L305V/K337A-FVII, V158D/E296V/M298Q/L305V-FVII, V158D/E296V/M298Q/K337A-FVII, V158D/E296V/M298Q/L305WK337A-FVII, K157A-FVII, E296V-FVII, E296V/M298Q-FVII, V158D/E296V-FVII, V158D/M298K-FVII, and S336G-FVII, L305V/K337A-FVII, L305V/V158D-FVII, L305V/E296V-FVII, L305V/M298Q-FVII, L305V/V158T-FVII, L305WK337A/V158T-FVII, L305V/K337A/M298Q-FVII, L305V/K337A/E296V-FVII, L305WK337A/V158D-FVII, L305V/V158D/M298Q-FVII, L305V/V158D/E296V-FVII, L305V/V158T/M298Q-FVII, L305V/V158T/E296V-FVII, L305V/E296V/M298Q-FVII, L305V/V158D/E296V/M298Q-FVII, L305V/V158T/E296V/M298Q-FVII, L305V/V158T/K337A/M298Q-FVII, L305V/V158T/E296WK337A-FVII, L305V/V158D/K337A/M298Q-FVII, L305V/V158D/E296WK337A-FVII, L305V/V158D/E296V/M298Q/K337A-FVII, L305V/V158T/E296V/M298Q/K337A-FVII, S314E/K316H-FVII, S314E/K316Q-FVII, S314E/L305V-FVII, S314E/K337A-FVII, S314E/V158D-FVII, S314E/E296V-FVII, S314E/M298Q-FVII, S314E/V158T-FVII, K316H/L305V-FVII, K316H/K337A-FVII, K316H/V158D-FVII, K316H/E296V-FVII, K316H/M298Q-FVII, K316H/V158T-FVII, K316Q/L305V-FVII, K316Q/K337A-FVII, K316Q/V158D-FVII, K316Q/E296V-FVII, K316Q/M298Q-FVII, K316Q/V158T-FVII, S314E/L305V/K337A-FVII, S314E/L305V/V158D-FVII, S314E/L305V/E296V-FVII, S314E/L305V/M298Q-FVII, S314E/L305V/V158T-FVII, S314E/L305V/K337A/V158T-FVII, S314E/L305V/K337A/M298Q-FVII, S314E/L305WK337A/E296V-FVII, S314E/L305V/K337A/V158D-FVII, S314E/L305V/V158D/M298Q-FVII, S314E/L305V/V158D/E296V-FVII, S314E/L305V/V158T/M298Q-FVII, S314E/L305V/V158T/E296V-FVII, S314E/L305V/E296V/M298Q-FVII, S314E/L305V/V158D/E296V/M298Q-FVII, S314E/L305V/V158T/E296V/M298Q-FVII, S314E/L305V/V158T/K337A/M298Q-FVII, S314E/L305V/V158T/E296WK337A-FVII, S314E/L305V/V158D/K337A/M298Q-FVII, S314E/L305V/V158D/E296V/K337A-FVII, S314E/L305V/V158D/E296V/M298Q/K337A-FVII, S314E/L305V/V158T/E296V/M298Q/K337A-FVII, K316H/L305V/K337A-FVII, K316H/L305V/V158D-FVII, K316H/L305V/E296V-FVII, K316H/L305V/M298Q-FVII, K316H/L305V/V158T-FVII, K316H/L305V/K337A/V158T-FVII, K316H/L305V/K337A/M298Q-FVII, K316H/L305V/K337A/E296V-FVII, K316H/L305V/K337A/V158D-FVII, K316H/L305V/V158D/M298Q-FVII, K316H/L305V/V158D/E296V-FVII, K316H/L305V/V158T/M298Q-FVII, K316H/L305V/V158T/E296V-FVII, K316H/L305V/E296V/M298Q-FVII, K316H/L305V/V158D/E296V/M298Q-FVII, K316H/L305V/V158T/E296V/M298Q-FVII, K316H/L305V/V158T/K337A/M298Q-FVII, K316H/L305V/V158T/E296V/K337A-FVII, K316H/L305V/V158D/K337A/M298Q-FVII, K316H/L305V/V158D/E296WK337A-FVII, K316H/L305V/V158D/E296V/M298Q/K337A-FVII, K316H/L305V/V158T/E296V/M298Q/K337A-FVII, K316Q/L305V/K337A-FVII, K316Q/L305V/V158D-FVII, K316Q/L305V/E296V-FVII, K316Q/L305V/M298Q-FVII, K316Q/L305V/V158T-FVII, K316Q/L305V/K337A/V158T-FVII, K316Q/L305WK337A/M298Q-FVII, K316Q/L305V/K337A/E296V-FVII, K316Q/L305V/K337A/V158D-FVII, K316Q/L305V/V158D/M298Q-FVII, K316Q/L305V/V158D/E296V-FVII, K316Q/L305V/V158T/M298Q-FVII, K316Q/L305V/V158T/E296V-FVII, K316Q/L305V/E296V/M298Q-FVII, K316Q/L305V/V158D/E296V/M298Q-FVII, K316Q/L305V/V158T/E296V/M298Q-FVII, K316Q/L305V/V158T/K337A/M298Q-FVII, K316Q/L305V/V158T/E296V/K337A-FVII, K316Q/L305V/V158D/K337A/M298Q-FVII, K316Q/L305V/V158D/E296V/K337A-FVII, K316Q/L305V/V158D/E296V/M298Q/K337A-FVII, K316Q/L305V/V158T/E296V/M298Q/K337A-FVII, F374Y/K337A-FVII, F374Y/V158D-FVII, F374Y/E296V-FVII, F374Y/M298Q-FVII, F374Y/V158T-FVII, F374Y/S314E-FVII, F374Y/L305V-FVII, F374Y/L305V/K337A-FVII, F374Y/L305V/V158D-FVII, F374Y/L305V/E296V-FVII, F374Y/L305V/M298Q-FVII, F374Y/L305V/V158T-FVII, F374Y/L305V/S314E-FVII, F374Y/K337A/S314E-FVII, F374Y/K337A/V158T-FVII, F374Y/K337A/M298Q-FVII, F374Y/K337A/E296V-FVII, F374Y/K337A/V158D-FVII, F374Y/V158D/S314E-FVII, F374Y/V158D/M298Q-FVII, F374Y/V158D/E296V-FVII, F374Y/V158T/S314E-FVII, F374Y/V158T/M298Q-FVII, F374Y/V158T/E296V-FVII, F374Y/E296V/S314E-FVII, F374Y/S314E/M298Q-FVII, F374Y/E296V/M298Q-FVII, F374Y/L305V/K337A/V158D-FVII, F374Y/L305V/K337A/E296V-FVII, F374Y/L305V/K337A/M298Q-FVII, F374Y/L305WK337A/V158T-FVII, F374Y/L305V/K337A/S314E-FVII, F374Y/L305V/V158D/E296V-FVII, F374Y/L305V/V158D/M298Q-FVII, F374Y/L305V/V158D/S314E-FVII, F374Y/L305V/E296V/M298Q-FVII, F374Y/L305V/E296V/V158T-FVII, F374Y/L305V/E296V/S314E-FVII, F374Y/L305V/M298Q/V158T-FVII, F374Y/L305V/M298Q/S314E-FVII, F374Y/L305V/V158T/S314E-FVII, F374Y/K337A/S314E/V158T-FVII, F374Y/K337A/S314E/M298Q-FVII, F374Y/K337A/S314E/E296V-FVII, F374Y/K337A/S314E/V158D-FVII, F374Y/K337A/V158T/M298Q-FVII, F374Y/K337A/V158T/E296V-FVII, F374Y/K337A/M298Q/E296V-FVII, F374Y/K337A/M298Q/V158D-FVII, F374Y/K337A/E296V/V158D-FVII, F374Y/V158D/S314E/M298Q-FVII, F374Y/V158D/S314E/E296V-FVII, F374Y/V158D/M298Q/E296V-FVII, F374Y/V158T/S314E/E296V-FVII, F374Y/V158T/S314E/M298Q-FVII, F374Y/V158T/M298Q/E296V-FVII, F374Y/E296V/S314E/M298Q-FVII, F374Y/L305V/M298Q/K337A/S314E-FVII, F374Y/L305V/E296V/K337A/S314E-FVII, F374Y/E296V/M298Q/K337A/S314E-FVII, F374Y/L305V/E296V/M298Q/K337A-FVII, F374Y/L305V/E296V/M298Q/S314E-FVII, F374Y/V158D/E296V/M298Q/K337A-FVII, F374Y/V158D/E296V/M298Q/S314E-FVII, F374Y/L305V/V158D/K337A/S314E-FVII, F374Y/V158D/M298Q/K337A/S314E-FVII, F374Y/V158D/E296V/K337A/S314E-FVII, F374Y/L305V/V158D/E296V/M298Q-FVII, F374Y/L305V/V158D/M298Q/K337A-FVII, F374Y/L305V/V158D/E296V/K337A-FVII, F374Y/L305V/V158D/M298Q/S314E-FVII, F374Y/L305V/V158D/E296V/S314E-FVII, F374Y/V158T/E296V/M298Q/K337A-FVII, F374Y/V158T/E296V/M298Q/S314E-FVII, F374Y/L305V/V158T/K337A/S314E-FVII, F374Y/V158T/M298Q/K337A/S314E-FVII, F374Y/V158T/E296V/K337A/S314E-FVII, F374Y/L305V/V158T/E296V/M298Q-FVII, F374Y/L305V/V158T/M298Q/K337A-FVII, F374Y/L305V/V158T/E296V/K337A-FVII, F374Y/L305V/V158T/M298Q/S314E-FVII, F374Y/L305V/V158T/E296V/S314E-FVII, F374Y/E296V/M298Q/K337A/V158T/S314E-FVII, F374Y/V158D/E296V/M298Q/K337A/S314E-FVII, F374Y/L305V/V158D/E296V/M298Q/S314E-FVII, F374Y/L305V/E296V/M298Q/V158T/S314E-FVII, F374Y/L305V/E296V/M298Q/K337A/V158T-FVII, F374Y/L305V/E296WK337A/V158T/S314E-FVII, F374Y/L305V/M298Q/K337A/V158T/S314E-FVII, F374Y/L305V/V158D/E296V/M298Q/K337A-FVII, F374Y/L305V/V158D/E296V/K337A/S314E-FVII, F374Y/L305V/V158D/M298Q/K337A/S314E-FVII, F374Y/L305V/E296V/M298Q/K337A/V158T/S314E-FVII, F374Y/L305V/V158D/E296V/M298Q/K337A/S314E-FVII, S52A-Factor VII, S60A-Factor VII; R152E-Factor VII, S344A-Factor VII, T106N-FVII, K143N/N145T-FVII, V253N-FVII, R290N/A292T-FVII, G291 N-FVII, R315N/V317T-FVII, K143N/N145T/R315N/V317T-FVII; and FVII having substitutions, additions or deletions in the amino acid sequence from 233Thr to 240Asn; FVII having substitutions, additions or deletions in the amino acid sequence from 304Arg to 329Cys; and FVII having substitutions, additions or deletions in the amino acid sequence from 153Ile to 223Arg.

Examples of Factor VII variants having substantially reduced or modified biological activity relative to wild-type Factor VII include, S344A-FVIIa (Kazama et al., J. Biol. Chem. 270:66-72, 1995), FFR-FVIIa (Holst et al., Eur. J. Vasc. Endovasc. Surg. 15:515-520, 1998), and Factor VIIa lacking the Gla domain, (Nicolaisen et al., FEBS Letts. 317:245-249, 1993), as well as completely inactivated Factor VIIa as disclosed in International Application No. WO 92/15686, all of which are incorporated herein by reference.

The term “PEGylated human Factor VIIa” means human Factor VIIa, having a PEG molecule conjugated to a human Factor VIIa polypeptide. It is to be understood, that the PEG molecule may be attached to any part of the Factor VIIa polypeptide including any amino acid residue or carbohydrate moiety of the Factor VIIa polypeptide. The term “cysteine-PEGylated human Factor VIIa” means Factor VIIa having a PEG molecule conjugated to a sulfhydryl group of a cysteine introduced in human Factor VIIa.

The biological activity of Factor VIIa in blood clotting derives from its ability to (i) bind to tissue factor (TF) and (ii) catalyze the proteolytic cleavage of Factor IX or Factor X to produce activated Factor IX or X (Factor IXa or Xa, respectively). For purposes of the invention, Factor VIIa biological activity may be quantified by measuring the ability of a preparation to promote blood clotting using Factor VII-deficient plasma and thromboplastin, as described, e.g., in U.S. Pat. No. 5,997,864. In this assay, biological activity is expressed as the reduction in clotting time relative to a control sample and is converted to “Factor VII units” by comparison with a pooled human serum standard containing 1 unit/ml Factor VII activity. Alternatively, Factor VIIa biological activity may be quantified by (i) measuring the ability of Factor VIIa to produce of Factor Xa in a system comprising TF embedded in a lipid membrane and Factor X. (Persson et al., J. Biol. Chem. 272:19919-19924, 1997); (ii) measuring Factor X hydrolysis in an aqueous system; (iii) measuring its physical binding to TF using an instrument based on surface plasmon resonance (Persson, FEBS Letts. 413:359-363, 1997) and (iv) measuring hydrolysis of a synthetic substrate.

Factor VII have been implicated in the treatment of disease related to coagulation, and biological active Factor VII compounds in particular have been implicated in the treatment of hemophiliacs, hemophiliacs with inhibitors to Factor VIII and IX, patients with thrombocytopenia, patients with thrombocytopathies, such as Glanzmann's thrombastenia platelet release defect and strorage pool defects, patient with von Willebrand's disease, patients with liver disease and bleeding problems associated with traumas or surgery. Biologically inactive Factor VII compounds have been implicated in the treatment of patients being in hypercoagluable states, such as patients with sepsis, deep-vein thrombosis, patients in risk of myocardial infections or thrombotic stroke, pulmonary embolism, patients with acute coronary syndromes, patients undergoing coronary cardiac, prevention of cardiac events and restenosis for patient receiving angioplasty, patient with peripheral vascular diseases, and acute respiratory distress syndrome. In one embodiment, the invention thus provides a method for the treatment of the above mentioned diseases or states, the method comprising administering to a subject in need thereof a therapeutically effective amount of a Factor VII compound conjugate according to the present invention.

In another embodiment, the invention provides the use of a Factor VII conjugate according to the present invention in the manufacture of a medicament used in the treatment of the above mentioned diseases or states.

Factor VII variants having substantially the same or improved biological activity relative to wild-type Factor VIIa encompass those that exhibit at least about 25%, preferably at least about 50%, more preferably at least about 75% and most preferably at least about 90% of the specific activity of Factor VIIa that has been produced in the same cell type, when tested in one or more of a clotting assay, proteolysis assay, or TF binding assay as described above. Factor VII variants having substantially reduced biological activity relative to wild-type Factor VIIa are those that exhibit less than about 25%, preferably less than about 10%, more preferably less than about 5% and most preferably less than about 1% of the specific activity of wild-type Factor VIIa that has been produced in the same cell type when tested in one or more of a clotting assay, proteolysis assay, or TF binding assay as described above. Factor VII variants having a modified biological activity relative to wild-type Factor VII include, without limitation, Factor VII variants that exhibit TF-independent Factor X proteolytic activity and those that bind TF but do not cleave Factor X.

In the present specification, amino acids are represented using abbreviations, as indicated in table 1, approved by IUPAC-IUB Commission on Biochemical Nomenclature (CBN). Amino acid and the like having isomers represented by name or the following abbreviations are in natural L-form unless otherwise indicated. Further, the left and right ends of an amino acid sequence of a peptide are, respectively, the N- and C-termini unless otherwise specified.

TABLE 1
Abbreviations for amino acids:

| | | One-letter code |
|---|---|---|
| | | (to be read in |
| Amino acid | Tree-letter code | context) |
| Glycine | Gly | G |
| Proline | Pro | P |
| Alanine | Ala | A |
| Valine | Val | V |
| Leucine | Leu | L |
| Isoleucine | Ile | I |
| Methionine | Met | M |
| Cysteine | Cys | C |
| Phenylalanine | Phe | F |
| Tyrosine | Tyr | Y |
| Tryptophan | Trp | W |
| Histidine | His | H |
| Lysine | Lys | K |
| Arginine | Arg | R |
| Glutamine | Gln | Q |
| Asparagine | Asn | N |
| Glutamic Acid | Glu | E |
| Aspartic Acid | Asp | D |
| Serine | Ser | S |
| Threonine | Thr | T |

The invention also relates to a method of preparing FVII related polypeptides or variants as mentioned above. FVII related polypeptides or variants may be produced by recombinant DNA techniques. To this end, DNA sequences encoding human FVII related polypeptides or FVII variants may be isolated by preparing a genomic or cDNA library and screening for DNA sequences coding for all or part of the protein by hybridization using synthetic oligonucleotide probes in accordance with standard techniques (cf. Sambrook et al., Molecular Cloning: A Laboratory Manual, Cold Spring Harbor Laboratory, Cold Spring Harbor, N.Y., 1989). For the present purpose, the DNA sequence encoding the protein is preferably of human origin, i.e. derived from a human genomic DNA or cDNA library.

The DNA sequences encoding the human FVII related polypeptides or FVII variants may also be prepared synthetically by established standard methods, e.g. the phosphoamidite method described by Beaucage and Caruthers, Tetrahedron Letters 22 (1981), 1859-1869, or the method described by Matthes et al., EMBO Journal3 (1984), 801-805. According to the phosphoramidite method, oligonucleotides are synthesized, e.g. in an automatic DNA synthesizer, purified, annealed, ligated and cloned in suitable vectors.

The DNA sequences may also be prepared by polymerase chain reaction using specific primers, for instance as described in U.S. Pat. No. 4,683,202, Saiki et al., Science 239 (1988), 487-491, or Sambrook et al., supra.

The DNA sequences encoding the FVII related polypeptides or FVII variants are usually inserted into a recombinant vector which may be any vector, which may conveniently be subjected to recombinant DNA procedures, and the choice of vector will often depend on the host cell into which it is to be introduced. Thus, the vector may be an autonomously replicating vector, i.e. a vector, which exists as an extrachromosomal entity, the replication of which is independent of chromosomal replication, e.g. a plasmid. Alternatively, the vector may be one which, when introduced into a host cell, is integrated into the host cell genome and replicated together with the chromosome(s) into which it has been integrated.

The vector is preferably an expression vector in which the DNA sequence encoding the FVII related polypeptides or FVII variants is operably linked to additional segments required for transcription of the DNA. In general, the expression vector is derived from plasmid or viral DNA, or may contain elements of both. The term, “operably linked” indicates that the segments are arranged so that they function in concert for their intended purposes, e.g. transcription initiates in a promoter and proceeds through the DNA sequence coding for the polypeptide.

The promoter may be any DNA sequence, which shows transcriptional activity in the host cell of choice and may be derived from genes encoding proteins either homologous or heterologous to the host cell.

Examples of suitable promoters for directing the transcription of the DNA encoding the human FVII polypeptide in mammalian cells are the SV40 promoter (Subramani et al., Mol. Cell Biol. 1 (1981), 854-864), the MT-1 (metallothionein gene) promoter (Palmiter et al., Science 222 (1983), 809-814), the CMV promoter (Boshart et al., Cell 41:521-530, 1985) or the adenovirus 2 major late promoter (Kaufman and Sharp, Mol. Cell. Biol, 2:1304-1319, 1982).

An example of a suitable promoter for use in insect cells is the polyhedrin promoter (U.S. Pat. No. 4,745,051; Vasuvedan et al., FEBS Lett. 311, (1992) 7-11), the P10 promoter (J. M. Vlak et al., J. Gen. Virology 69, 1988, pp. 765-776), the Autographa californica polyhedrosis virus basic protein promoter (EP 397 485), the baculovirus immediate early gene 1 promoter (U.S. Pat. No. 5,155,037; U.S. Pat. No. 5,162,222), or the baculovirus 39K delayed-early gene promoter (U.S. Pat. No. 5,155,037; U.S. Pat. No. 5,162,222).

Examples of suitable promoters for use in yeast host cells include promoters from yeast glycolytic genes (Hitzeman et al., J. Biol. Chem. 255 (1980), 12073-12080; Alber and Kawasaki, J. Mol. Appl. Gen. 1 (1982), 419-434) or alcohol dehydrogenase genes (Young et al., in Genetic Engineering of Microorganisms for Chemicals (Hollaender et al, eds.), Plenum Press, New York, 1982), or the TPI1 (U.S. Pat. No. 4,599,311) or ADH2-4-c (Russell et al., Nature 304 (1983), 652-654) promoters.

Examples of suitable promoters for use in filamentous fungus host cells are, for instance, the ADH3 promoter (McKnight et al., The EMBO J. 4 (1985), 2093-2099) or the tpiA promoter. Examples of other useful promoters are those derived from the gene encoding A. oryzae TAKA amylase, Rhizomucor miehei aspartic proteinase, A. niger neutral α-amylase, A. niger acid stable α-amylase, A. niger or A. awamori glucoamylase (gluA), Rhizomucor miehei lipase, A. oryzae alkaline protease, A. oryzae triose phosphate isomerase or A. nidulans acetamidase. Preferred are the TAKA-amylase and gluA promoters. Suitable promoters are mentioned in, e.g. EP 238 023 and EP 383 779.

The DNA sequences encoding the FVII related polypeptides or FVII variants may also, if necessary, be operably connected to a suitable terminator, such as the human growth hormone terminator (Palmiter et al., Science 222, 1983, pp. 809-814) or the TPI1 (Alber and Kawasaki, J. Mol. Appl. Gen. 1, 1982, pp. 419-434) or ADH3 (McKnight et al., The EMBO J. 4, 1985, pp. 2093-2099) terminators. The vector may also contain a set of RNA splice sites located downstream from the promoter and upstream from the insertion site for the FVII sequence itself. Preferred RNA splice sites may be obtained from adenovirus and/or immunoglobulin genes. Also contained in the expression vectors is a polyadenylation signal located downstream of the insertion site. Particularly preferred polyadenylation signals include the early or late polyadenylation signal from SV40 (Kaufman and Sharp, ibid.), the polyadenylation signal from the adenovirus 5 E1b region, the human growth hormone gene terminator (DeNoto et al. Nuc. Acids Res. 9:3719-3730, 1981) or the polyadenylation signal from the human FVII gene or the bovine FVII gene. The expression vectors may also include a noncoding viral leader sequence, such as the adenovirus 2 tripartite leader, located between the promoter and the RNA splice sites; and enhancer sequences, such as the SV40 enhancer.

The recombinant vector may further comprise a DNA sequence enabling the vector to replicate in the host cell in question. An example of such a sequence (when the host cell is a mammalian cell) is the SV40 origin of replication.

When the host cell is a yeast cell, suitable sequences enabling the vector to replicate are the yeast plasmid 2μ replication genes REP 1-3 and origin of replication.

The vector may also comprise a selectable marker, e.g. a gene the product of which complements a defect in the host cell, such as the gene coding for dihydrofolate reductase (DHFR) or the Schizosaccharomyces pombe TPI gene (described by P. R. Russell, Gene 40, 1985, pp. 125-130), or one which confers resistance to a drug, e.g. ampicillin, kanamycin, tetracyclin, chloramphenicol, neomycin, hygromycin or methotrexate. For filamentous fungi, selectable markers include amdS, pyrG, argB, niaD or sC.

To direct the human FVII related polypeptides or FVII variants of the present invention into the secretory pathway of the host cells, a secretory signal sequence (also known as a leader sequence, prepro sequence or pre sequence) may be provided in the recombinant vector. The secretory signal sequence is joined to the DNA sequences encoding the FVII related polypeptides or FVII variants in the correct reading frame. Secretory signal sequences are commonly positioned 5′ to the DNA sequence encoding the peptide. The secretory signal sequence may be that, normally associated with the protein or may be from a gene encoding another secreted protein.

For secretion from yeast cells, the secretory signal sequence may encode any signal peptide, which ensures efficient direction of the expressed FVII related polypeptides or FVII variants into the secretory pathway of the cell. The signal peptide may be naturally occurring signal peptide, or a functional part thereof, or it may be a synthetic peptide. Suitable signal peptides have been found to be the α-factor signal peptide (cf. U.S. Pat. No. 4,870,008), the signal peptide of mouse salivary amylase (cf. O. Hagenbuchle et al., Nature 289, 1981, pp. 643-646), a modified carboxypeptidase signal peptide (cf. L. A. Valls et al., Cell 48, 1987, pp. 887-897), the yeast BAR1 signal peptide (cf. WO 87/02670), or the yeast aspartic protease 3 (YAP3) signal peptide (cf. M. Egel-Mitani et al., Yeast 6, 1990, pp. 127-137).

For efficient secretion in yeast, a sequence encoding a leader peptide may also be inserted downstream of the signal sequence and upstream of the DNA sequence encoding the FVII related polypeptides or FVII variants. The function of the leader peptide is to allow the expressed peptide to be directed from the endoplasmic reticulum to the Golgi apparatus and further to a secretory vesicle for secretion into the culture medium (i.e. exportation of the FVII related polypeptides or FVII variants across the cell wall or at least through the cellular membrane into the periplasmic space of the yeast cell). The leader peptide may be the yeast alpha-factor leader (the use of which is described in e.g. U.S. Pat. No. 4,546,082, U.S. Pat. No. 4,870,008, EP 16 201, EP 123 294, EP 123 544 and EP 163 529). Alternatively, the leader peptide may be a synthetic leader peptide, which is to say a leader peptide not found in nature. Synthetic leader peptides may, for instance, be constructed as described in WO 89/02463 or WO 92/11378.

For use in filamentous fungi, the signal peptide may conveniently be derived from a gene encoding an Aspergillus sp. amylase or glucoamylase, a gene encoding a Rhizomucor miehei lipase or protease or a Humicola lanuginosa lipase. The signal peptide is preferably derived from a gene encoding A. oryzae TAKA amylase, A. niger neutral α-amylase, A. niger acid-stable amylase, or A. niger glucoamylase. Suitable signal peptides are disclosed in, e.g. EP 238 023 and EP 215 594.

For use in insect cells, the signal peptide may conveniently be derived from an insect gene (cf. WO 90/05783), such as the lepidopteran Manduca sexta adipokinetic hormone precursor signal peptide (cf. U.S. Pat. No. 5,023,328).

The procedures used to ligate the DNA sequences coding for the FVII related polypeptides or FVII variants, the promoter and optionally the terminator and/or secretory signal sequence, respectively, and to insert them into suitable vectors containing the information necessary for replication, are well known to persons skilled in the art (cf., for instance, Sambrook et al., Molecular Cloning: A Laboratory Manual, Cold Spring Harbor, N.Y., 1989).

Methods of transfecting mammalian cells and expressing DNA sequences introduced in the cells are described in e.g. Kaufman and Sharp, J. Mol. Biol. 159 (1982), 601-621; Southern and Berg, J. Mol. Appl. Genet. 1 (1982), 327-341; Loyter et al., Proc. Natl. Acad. Sci. USA 79 (1982), 422-426; Wigler et al., Cell 14 (1978), 725; Corsaro and Pearson, Somatic Cell Genetics 7 (1981), 603, Graham and van der Eb, Virology 52 (1973), 456; and Neumann et al., EMBO J. 1 (1982), 841-845.

Selectable markers may be introduced into the cell on a separate plasmid at the same time as the gene of interest, or they may be introduced on the same plasmid. If on the same plasmid, the selectable marker and the gene of interest may be under the control of different promoters or the same promoter, the latter arrangement producing a dicistronic message. Constructs of this type are known in the art (for example, Levinson and Simonsen, U.S. Pat. No. 4,713,339). It may also be advantageous to add additional DNA, known as “carrier DNA,” to the mixture that is introduced into the cells.

After the cells have taken up the DNA, they are grown in an appropriate growth medium, typically 1-2 days, to begin expressing the gene of interest. As used herein the term “appropriate growth medium” means a medium containing nutrients and other components required for the growth of cells and the expression of the FVII related polypeptides or FVII variants of interest. Media generally include a carbon source, a nitrogen source, essential amino acids, essential sugars, vitamins, salts, phospholipids, protein and growth factors. For production of gamma-carboxylated proteins, the medium will contain vitamin K, preferably at a concentration of about 0.1 μg/ml to about 5 μg/ml. Drug selection is then applied to select for the growth of cells that are expressing the selectable marker in a stable fashion. For cells that have been transfected with an amplifiable selectable marker the drug concentration may be increased to select for an increased copy number of the cloned sequences, thereby increasing expression levels. Clones of stably transfected cells are then screened for expression of the human FVII polypeptide of interest.

The host cell into which the DNA sequences encoding the FVII related polypeptides or FVII variants is introduced may be any cell, which is capable of producing the posttranslational modified FVII related polypeptides or FVII variants and includes yeast, fungi and higher eucaryotic cells.

Examples of mammalian cell lines for use in the present invention are the COS-1 (ATCC CRL 1650), baby hamster kidney (BHK) and 293 (ATCC CRL 1573; Graham et al., J. Gen. Virol. 36:59-72, 1977) cell lines. A preferred BHK cell line is the tk⁻ ts13 BHK cell line (Waechter and Baserga, Proc. Natl. Acad. Sci. USA 79:1106-1110, 1982, incorporated herein by reference), hereinafter referred to as BHK 570 cells. The BHK 570 cell line has been deposited with the American Type Culture Collection, 12301 Parklawn Dr., Rockville, Md. 20852, under ATCC accession number CRL 10314. A tk⁻ ts13 BHK cell line is also available from the ATCC under accession number CRL 1632. In addition, a number of other cell lines may be used within the present invention, including Rat Hep I (Rat hepatoma; ATCC CRL 1600), Rat Hep II (Rat hepatoma; ATCC CRL 1548), TCMK (ATCC CCL 139), Human lung (ATCC HB 8065), NCTC 1469 (ATCC CCL 9.1), CHO (ATCC CCL 61) and DUKX cells (Urlaub and Chasin, Proc. Natl. Acad. Sci. USA 77:4216-4220, 1980).

Examples of suitable yeasts cells include cells of Saccharomyces spp. or Schizosaccharomyces spp., in particular strains of Saccharomyces cerevisiae or Saccharomyces kluyveri. Methods for transforming yeast cells with heterologous DNA and producing heterologous polypeptides there from are described, e.g. in U.S. Pat. No. 4,599,311, U.S. Pat. No. 4,931,373, U.S. Pat. Nos. 4,870,008, 5,037,743, and U.S. Pat. No. 4,845,075, all of which are hereby incorporated by reference. Transformed cells are selected by a phenotype determined by a selectable marker, commonly drug resistance or the ability to grow in the absence of a particular nutrient, e.g. leucine. A preferred vector for use in yeast is the POT1 vector disclosed in U.S. Pat. No. 4,931,373. The DNA sequences encoding the human FVII polypeptides may be preceded by a signal sequence and optionally a leader sequence, e.g. as described above. Further examples of suitable yeast cells are strains of Kluyveromyces, such as K. lactis, Hansenula, e.g. H. polymorpha, or Pichia, e.g. P. pastoris (cf. Gleeson et al., J. Gen. Microbiol. 132, 1986, pp. 3459-3465; U.S. Pat. No. 4,882,279).

Examples of other fungal cells are cells of filamentous fungi, e.g. Aspergillus spp., Neurospora spp., Fusarium spp. or Trichoderma spp., in particular strains of A. oryzae, A. nidulans or A. niger. The use of Aspergillus spp. for the expression of proteins is described in, e.g., EP 272 277, EP 238 023, EP 184 438 The transformation of F oxysporum may, for instance, be carried out as described by Malardier et al., 1989, Gene 78: 147-156. The transformation of Trichoderma spp. may be performed for instance as described in EP 244 234.

When a filamentous fungus is used as the host cell, it may be transformed with the DNA construct of the invention, conveniently by integrating the DNA construct in the host chromosome to obtain a recombinant host cell. This integration is generally considered to be an advantage as the DNA sequence is more likely to be stably maintained in the cell. Integration of the DNA constructs into the host chromosome may be performed according to conventional methods, e.g. by homologous or heterologous recombination.

Transformation of insect cells and production of heterologous polypeptides therein may be performed as described in U.S. Pat. No. 4,745,051; U.S. Pat. No. 4,879,236; U.S. Pat. Nos. 5,155,037; 5,162,222; EP 397,485) all of which are incorporated herein by reference. The insect cell line used as the host may suitably be a Lepidoptera cell line, such as Spodoptera frugiperda cells or Trichoplusia ni cells (cf. U.S. Pat. No. 5,077,214). Culture conditions may suitably be as described in, for instance, WO 89/01029 or WO 89/01028, or any of the aforementioned references.

The transformed or transfected host cell described above is then cultured in a suitable nutrient medium under conditions permitting expression of the FVII related polypeptides or FVII variants after which all or part of the resulting peptide may be recovered from the culture. The medium used to culture the cells may be any conventional medium suitable for growing the host cells, such as minimal or complex media containing appropriate supplements. Suitable media are available from commercial suppliers or may be prepared according to published recipes (e.g. in catalogues of the American Type Culture Collection). The FVII related polypeptides or FVII variants produced by the cells may then be recovered from the culture medium by conventional procedures including separating the host cells from the medium by centrifugation or filtration, precipitating the proteinaqueous components of the supernatant or filtrate by means of a salt, e.g. ammonium sulphate, purification by a variety of chromatographic procedures, e.g. ion exchange chromatography, gelfiltration chromatography, affinity chromatography, or the like, dependent on the type of polypeptide in question.

For the preparation of recombinant FVII related polypeptides or FVII variants, a cloned wild-type FVII DNA sequence is used. This sequence may be modified to encode a desired FVII variant. The complete nucleotide and amino acid sequences for human FVII are known. See U.S. Pat. No. 4,784,950, which is incorporated herein by reference, where the cloning and expression of recombinant human FVII is described. The bovine FVII sequence is described in Takeya et al., J. Biol. Chem., 263:14868-14872 (1988), which is incorporated by reference herein.

The amino acid sequence alterations may be accomplished by a variety of techniques. Modification of the DNA sequence may be by site-specific mutagenesis.

Techniques for site-specific mutagenesis are well known in the art and are described by, for example, Zoller and Smith (DNA 3:479-488, 1984). Thus, using the nucleotide and amino acid sequences of FVII, one may introduce the alterations of choice.

DNA sequences for use within the present invention will typically encode a pre-pro peptide at the amino-terminus of the FVII related polypeptides or FVII variants to obtain proper post-translational processing (e.g. gamma-carboxylation of glutamic acid residues) and secretion from the host cell. The pre-pro peptide may be that of FVII or another vitamin K-dependent plasma protein, such as factor IX, factor X, prothrombin, protein C or protein S. As will be appreciated by those skilled in the art, additional modifications can be made in the amino acid sequence of FVII. For example, FVII in the catalytic triad can also be modified in the activation cleavage site to inhibit the conversion of zymogen FVII into its activated two-chain form, as generally described in U.S. Pat. No. 5,288,629, incorporated herein by reference.

Within the present invention, transgenic animal technology may be employed to produce the FVII related polypeptides or FVII variants. It is preferred to produce the proteins within the mammary glands of a host female mammal. Expression in the mammary gland and subsequent secretion of the protein of interest into the milk overcomes many difficulties encountered in isolating proteins from other sources. Milk is readily collected, available in large quantities, and well characterized biochemically. Furthermore, the major milk proteins are present in milk at high concentrations (typically from about 1 to 15 g/l). From a commercial point of view, it is clearly preferable to use as the host a species that has a large milk yield. While smaller animals such as mice and rats can be used (and are preferred at the proof of principle stage), within the present invention it is preferred to use livestock mammals including, but not limited to, pigs, goats, sheep and cattle. Sheep are particularly preferred due to such factors as the previous history of transgenesis in this species, milk yield, cost and the ready availability of equipment for collecting sheep milk. See WIPO Publication WO 88/00239 for a comparison of factors influencing the choice of host species. It is generally desirable to select a breed of host animal that has been bred for dairy use, such as East Friesland sheep, or to introduce dairy stock by breeding of the transgenic line at a later date. In any event, animals of known, good health status should be used.

To obtain expression in the mammary gland, a transcription promoter from a milk protein gene is used. Milk protein genes include those genes encoding caseins (see U.S. Pat. No. 5,304,489, incorporated herein by reference), beta-lactoglobulin, alpha-lactalbumin, and whey acidic protein. The beta-lactoglobulin (BLG) promoter is preferred. In the case of the ovine beta-lactoglobulin gene, a region of at least the proximal 406 bp of 5′ flanking sequence of the gene will generally be used, although larger portions of the 5′ flanking sequence, up to about 5 kbp, are preferred, such as about 4.25 kbp DNA segment encompassing the 5′ flanking promoter and non-coding portion of the beta-lactoglobulin gene. See Whitelaw et al., Biochem J. 286: 31-39 (1992). Similar fragments of promoter DNA from other species are also suitable.

Other regions of the beta-lactoglobulin gene may also be incorporated in constructs, as may genomic regions of the gene to be expressed. It is generally accepted in the art that constructs lacking introns, for example, express poorly in comparison with those that contain such DNA sequences (see Brinster et al., Proc. Natl. Acad. Sci. USA 85: 836-840 (1988); Palmiter et al., Proc. Natl. Acad. Sci. USA 88: 478-482 (1991); Whitelaw et al., Transgenic Res. 1: 3-13 (1991); WO 89/01343; and WO 91/02318, each of which is incorporated herein by reference). In this regard, it is generally preferred, where possible, to use genomic sequences containing all or some of the native introns of a gene encoding the protein or polypeptide of interest, thus the further inclusion of at least some introns from, e.g, the beta-lactoglobulin gene, is preferred. One such region is a DNA segment which provides for intron splicing and RNA polyadenylation from the 3′ non-coding region of the ovine beta-lactoglobulin gene. When substituted for the natural 3′ non-coding sequences of a gene, this ovine beta-lactoglobulin segment can both enhance and stabilize expression levels of the protein or polypeptide of interest. Within other embodiments, the region surrounding the initiation ATG of the sequence encoding the FVII related polypeptides or FVII variants is replaced with corresponding sequences from a milk specific protein gene. Such replacement provides a putative tissue-specific initiation environment to enhance expression. It is convenient to replace the entire pre-pro sequence of the FVII related polypeptides or FVII variants and 5′ non-coding sequences with those of, for example, the BLG gene, although smaller regions may be replaced.

For expression of a FVII related polypeptides or FVII variants in transgenic animals, a DNA segment encoding the FVII related polypeptides or FVII variants is operably linked to additional DNA segments required for its expression to produce expression units. Such additional segments include the above-mentioned promoter, as well as sequences which provide for termination of transcription and polyadenylation of mRNA. The expression units will further include a DNA segment encoding a secretory signal sequence operably linked to the segment encoding the FVII related polypeptides or FVII variants. The secretory signal sequence may be a native secretory signal sequence of the human FVII polypeptide or may be that of another protein, such as a milk protein. See, for example, von Heinje, Nuc. Acids Res. 14: 4683-4690 (1986); and Meade et al., U.S. Pat. No. 4,873,316, which are incorporated herein by reference.

Construction of expression units for use in transgenic animals is conveniently carried out by inserting a sequence encoding the FVII related polypeptides or FVII variants into a plasmid or phage vector containing the additional DNA segments, although the expression unit may be constructed by essentially any sequence of ligations. It is particularly convenient to provide a vector containing a DNA segment encoding a milk protein and to replace the coding sequence for the milk protein with that of the human FVII polypeptide, thereby creating a gene fusion that includes the expression control sequences of the milk protein gene. In any event, cloning of the expression units in plasmids or other vectors facilitates the amplification of the FVII related polypeptides or FVII variants. Amplification is conveniently carried out in bacterial (e.g. E. coli) host cells, thus the vectors will typically include an origin of replication and a selectable marker functional in bacterial host cells.

The expression unit is then introduced into fertilized eggs (including early-stage embryos) of the chosen host species. Introduction of heterologous DNA can be accomplished by one of several routes, including microinjection (e.g. U.S. Pat. No. 4,873,191), retroviral infection (Jaenisch, Science 240: 1468-1474 (1988)) or site-directed integration using embryonic stem (ES) cells (reviewed by Bradley et al., Bio/Technology 10: 534-539 (1992)). The eggs are then implanted into the oviducts or uteri of pseudopregnant females and allowed to develop. Offspring carrying the introduced DNA in their germ line can pass the DNA on to their progeny in the normal, Mendelian fashion, allowing the development of transgenic herds.

General procedures for producing transgenic animals are known in the art. See, for example, Hogan et al., Manipulating the Mouse Embryo: A Laboratory Manual, Cold Spring Harbor Laboratory, 1986; Simons et al., Bio/Technology 6: 179-183 (1988); Wall et al., Biol. Reprod. 32: 645-651 (1985); Buhler et al., Bio/Technology 8: 140-143 (1990); Ebert et al., Bio/Technology 9: 835-838 (1991); Krimpenfort et al., Bio/Technology 9: 844-847 (1991); Wall et al., J. Cell. Biochem. 49:113-120 (1992); U.S. Pat. Nos. 4,873,191 and 4,873,316; WIPO publications WO 88/00239, WO 90/05188, WO 92/11757; and GB 87/00458, which are incorporated herein by reference. Techniques for introducing foreign DNA sequences into mammals and their germ cells were originally developed in the mouse. See, e.g., Gordon et al., Proc. Natl. Acad. Sci. USA 77: 7380-7384 (1980); Gordon and Ruddle, Science 214: 1244-1246 (1981); Palmiter and Brinster, Cell 41: 343-345 (1985); and Brinster et al., Proc. Natl. Acad. Sci. USA 82: 4438-4442 (1985). These techniques were subsequently adapted for use with larger animals, including livestock species (see e.g., WIPO publications WO 88/00239, WO 90/05188, and WO 92/11757; and Simons et al., Bio/Technology 6: 179-183 (1988). To summarize, in the most efficient route used to date in the generation of transgenic mice or livestock, several hundred linear molecules of the DNA of interest are injected into one of the pro-nuclei of a fertilized egg according to established techniques. Injection of DNA into the cytoplasm of a zygote can also be employed. Production in transgenic plants may also be employed. Expression may be generalized or directed to a particular organ, such as a tuber. See, Hiatt, Nature 344:469-479 (1990); Edelbaum et al., J. Interferon Res. 12:449-453 (1992); Sijmons et al., Bio/Technology 8:217-221 (1990); and European Patent Office Publication EP 255,378.

FVII related polypeptides or FVII variants produced according to the present invention may be purified by affinity chromatography on an anti-FVII antibody column. It is preferred that the immunoadsorption column comprise a high-specificity monoclonal antibody. The use of calcium-dependent monoclonal antibodies, as described by Wakabayashi et al., J. Biol. Chem., 261:11097-11108, (1986) and Thim et al., Biochem. 27: 7785-7793, (1988), incorporated by reference herein, is particularly preferred. Additional purification may be achieved by conventional chemical purification means, such as high performance liquid chromatography. Other methods of purification, including barium citrate precipitation, are known in the art, and may be applied to the purification of the FVII described herein (see, generally, Scopes, R., Protein Purification, Springer-Verlag, N.Y., 1982). Substantially pure FVII related polypeptides or FVII variants of at least about 90 to 95% homogeneity is preferred, and 98 to 99% or more homogeneity most preferred, for pharmaceutical uses. Once purified, partially or to homogeneity as desired, the FVII related polypeptides or FVII variants may then be used according to the present invention.

α-amino acid amides are, as mentioned previously, particular well-suited as a nucleophile in the methods of the present invention. In one embodiment, the invention thus provides compounds according to formula (I)

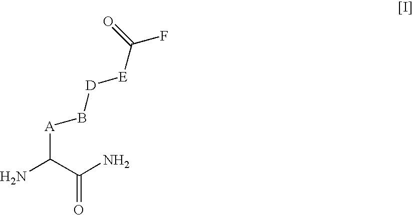

wherein A and E independently represent C₁₋₆alkylene, C₂₋₆alkenylene, C₂₋₆alkynylene or arylene, all of which may optionally be substituted with one or more substituents selected from halogen, amino, cyano and nitro;
B and D represents independently a valence bond, —O—, —S—, —NH—, —C(O)—NH— or —NH—C(O)—; and F represents hydrogen or C₁₋₆alkyl, C₂₋₆alkenyl, C₂₋₆alkynyl or aryl, all of which may optionally be substituted with one or more substituents selected from halogen, amino, cyano and nitro.
In principle the α-amino acid amides may in general comprise a sequence of amino acids as below:

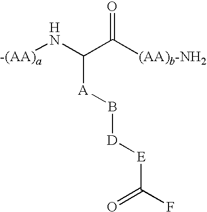

wherein AA represents any amino acid, a and b are any number including 0 and A, B, D, E and F has the meaning as described above.
Thus in the following the described compounds and general structures should be regarded as amino acid amides to be used either as a monomer or as a C-terminal part of a peptide, or as amino acid derivatives to be inserted internally in a sequence of amino acids, for addition to the P′.

In one embodiment, A and E independently represent C₁₋₆alkylene, such as methylene, ethylene, propylene, butylenes, pentylene or hexylene, or arylene, such as phenylene.

In one embodiment, F represents hydrogen or methyl, ethyl, propyl or butyl.

Particular examples of a compound of formula I include
(2S)-2-Amino-6-(4-oxo-4-phenylbutyrylamino)hexanoic acid
4-Acetyl-N-((5S)-5-amino-5-carbamoylpentyl)benzoic acid
(2S)-2-Amino-6-(4-oxo-4-(4-chlorophenylbutyrylamino)hexanoic acid
3-Acetyl-N-((5S)-5-amino-5-carbamoylpentyl)benzoic acid, and
2-Acetyl-N-((5S)-5-amino-5-carbamoylpentyl)benzoic acid

and each of them also as the amide derivative

In another embodiment, the invention provides compounds according to formula II

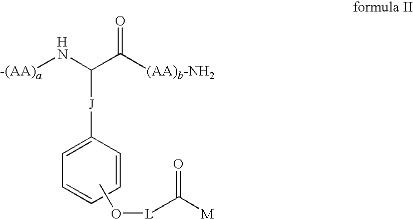

wherein J and L independently represent C₁₋₆alkylene, C₂₋₆alkenylene, C₂₋₆alkynylene or arylene, all of which may optionally be substituted with one or more substituents selected from halogen, amino, cyano and nitro;
and M represents hydrogen or C₁₋₆alkyl.

In one embodiment, J and L independently represent C₁₋₆alkylene, such as methylene, ethylene, propylene, butylenes, pentylene or hexylene, or arylene, such as phenylene.

In one embodiment, M represents hydrogen or methyl, ethyl, propyl or butyl.

In one embodiment, the compounds of formula II are selected from amongst
(2S)-Amino-3-[4-(2-oxopropoxy)phenyl]propionamide,
(2S)-Amino-3-[4-(2-oxobutoxy)phenyl]propionamide,
(2S)-Amino-3-[4-(2-oxopentoxy)phenyl]propionamide, and
(2S)-Amino-3-[4-(4-oxopentoxy)phenyl]propionamide
   and the respective acid derivatives.

In still another embodiment, the invention provides compounds according to formula III

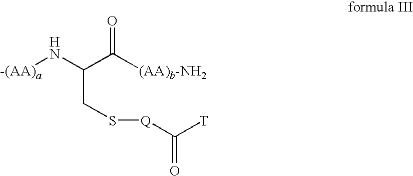

wherein Q represents represent C₁₋₆alkylene, C₂₋₆alkenylene, C₂₋₆alkynylene or arylene, all of which may optionally be substituted with one or more substituents selected from halogen, amino, cyano and nitro;
and T represents hydrogen or C₁₋₆alkyl.

In one embodiment, Q represents C₁₋₆alkylene, such as methylene, ethylene, propylene, butylenes, pentylene or hexylene, or arylene, such as phenylene. In one embodiment, T represents hydrogen or methyl, ethyl, propyl or butyl.

The nucleophile, e.g. the compound of the formula

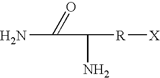

or the corresponding acid may either be acquired commercially or synthesized according to the following guidelines in general Methods A-F.

General Method (A):

A compound of the general formula

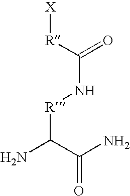

or the corresponding acid
wherein R″ and R′″ independently represents C₁₋₁₅alkylene, C₂₋₁₅alkenylene, C₂₋₁₅alkynylene, C₁₋₁₅heteroalkylene, C₂₋₁₅heteroalkenylene, C₂₋₁₅heteroalkynylene, wherein one or more homocyclic aromatic compound biradical or heterocyclic compound biradical may be inserted, may be prepared by a person skilled in the art, from a suitable amino acid methyl ester which is protected at the alpha-amino group by a suitable protecting group PG, known to a person skilled in the art and described in the literature e.g. in (e.g. T. W. Greene, P. G. M. Wuts, Protective groups in organic synthesis, 2^{nd} ed., 1991 John Wiley & Sons, Inc. New York)

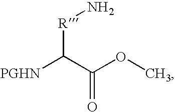

by an acylation method, e.g. using an suitable acid, in which X may or may not be protected by a suitable protective group, known to a person skilled in the art and described in the literature e.g. in (e.g. T. W. Greene, P. G. M. Wuts, Protective groups in organic synthesis, 2^{nd} ed., 1991 John Wiley & Sons, Inc. New York).

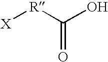

and a coupling reagent such as e.g. 1-hydroxybenzotriazole, 3,4-dihydro-3-hydroxybenzotriazin-4-one or 7-azabenzotriazole in combination with e.g. a carbodiimide such as e.g. diisopropylcarbodiimide or 1-(3-dimethylaminopropyl)-3-ethylcarbodiimide hydrochloride in the presence or absence of a suitable base such as e.g. triethylamine or ethyldiisopropylamine to form the ester of type

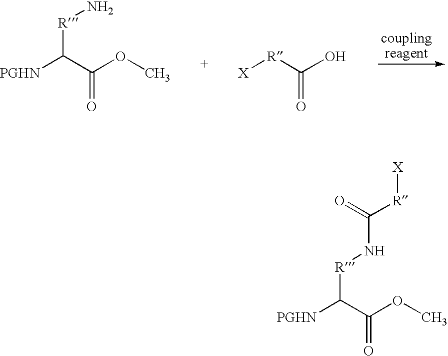

The ester may be transformed into the corresponding amide by reaction with e.g. ammonia in a suitable solvent or mixture of solvents such as e.g. water or N,N-dimethylformamide.

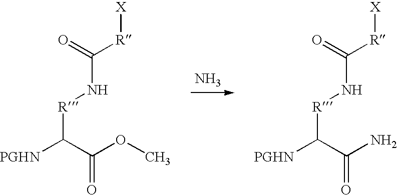

If the acid is the desired compound the ester is hydrolysed.

The removal of all protective groups may be performed in one or several steps by methods known to a person skilled in the art and described in the literature (e.g. T. W. Greene, P. G. M. Wuts, Protective groups in organic synthesis, 2^{nd} ed., 1991 John Wiley & Sons, Inc. New York).

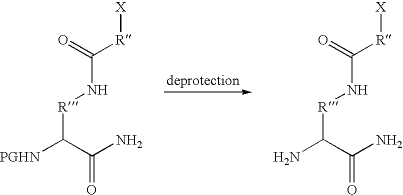

As defined in General Method (A)

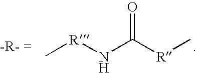

Amino acid methyl esters are generally commercially available, or they may be synthesized by well-known methods.

General Method (B):

A compound of the general formula

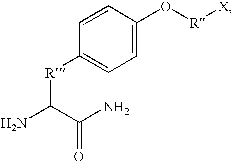

wherein R″ and R′″ are defined as above, may be prepared by a person skilled in the art, from a suitable amino acid methyl ester which is protected at the alpha-amino group by a suitable protecting group PG, known to a person skilled in the art and described in the literature e.g. in (e.g. T. W. Greene, P. G. M. Wuts, Protective groups in organic synthesis, 2^{nd} ed., 1991 John Wiley & Sons, Inc. New York)

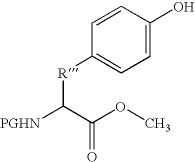

by an alkylation of the aromatic hydroxyl group, using an suitable alcohol, in which X may or may not be protected by a suitable protective group, known to a person skilled in the art and described in the literature e.g. in (e.g. T. W. Greene, P. G. M. Wuts, Protective groups in organic synthesis, 2^{nd} ed., 1991 John Wiley & Sons, Inc. New York)

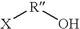

under conditions which effect alkylation, known to a person skilled in the art and described in the literature e.g. Mitsunobu conditions such as e.g. triphenylphosphine and diethyl azodicarboxylate to form the ester of type.

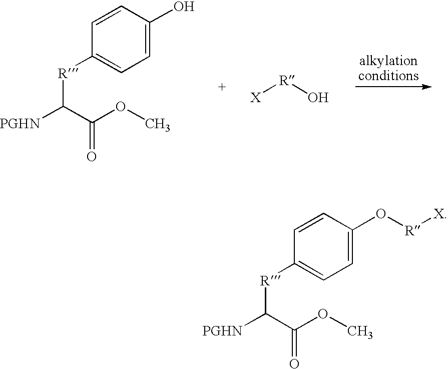

The ester may be transformed into the corresponding amide by reaction with e.g. ammonia in a suitable solvent or mixture of solvents such as e.g. water or N,N-dimethylformamide.

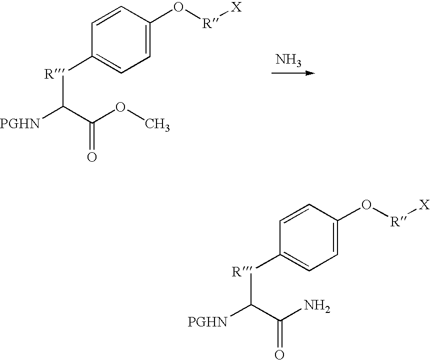

Or the ester is simply hydrolysed to the acid derivative.

The removal of all protective groups may be performed in one or several steps by methods known to a person skilled in the art and described in the literature (e.g. T. W. Greene, P. G. M. Wuts, Protective groups in organic synthesis, 2^{nd} ed., 1991 John Wiley & Sons, Inc. New York).

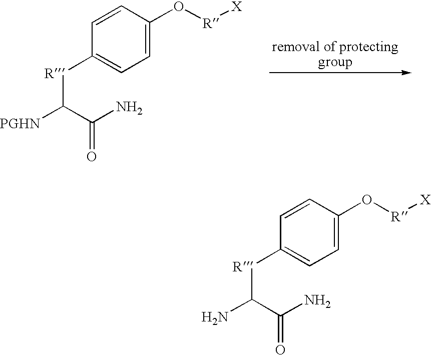

As defined in General Method (B)

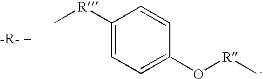

General Method (C):

A compound of the general formula

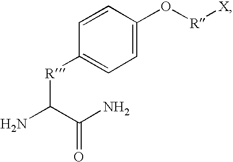

wherein R″ and R′″ are defined as above, may be prepared by a person skilled in the art, from a suitable amino acid methyl ester which is protected at the alpha-amino group by a suitable protecting group PG, known to a person skilled in the art and described in the literature e.g. in (e.g. T. W. Greene, P. G. M. Wuts, Protective groups in organic synthesis, 2^{nd} ed., 1991 John Wiley & Sons, Inc. New York)

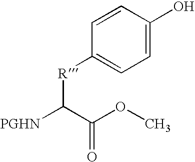

by an alkylation of the aromatic hydroxyl group, using an suitable alkylation reagent

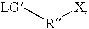

in which the anion of LG′ is a suitable leaving group such as halogenide or sulfonate and X may or may not be protected by a suitable protective group, known to a person skilled in the art and described in the literature e.g. in (e.g. T. W. Greene, P. G. M. Wuts, Protective groups in organic synthesis, 2^{nd} ed., 1991 John Wiley & Sons, Inc. New York). The reaction may take place under basic conditions, applying bases such as e.g. potassium carbonate, diazabicylo[5,4,0]undec-5-ene, or tert-butyltetramethyluanidine at a suitable temperature, typically between −78° C. and 200° C.

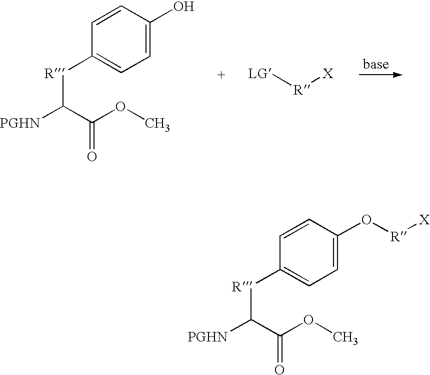

The ester may be transformed into the corresponding amide by reaction with e.g. ammonia in a suitable solvent or mixture of solvents such as e.g. water or N,N-dimethylformamide.

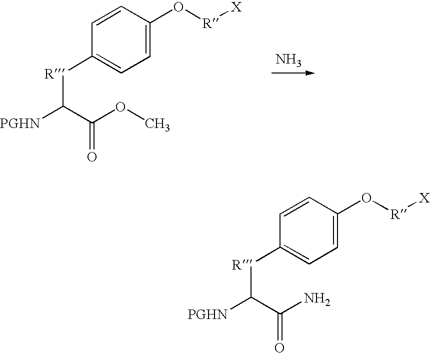

Or the ester is hydrolysed to obtain the acid derivative.

The removal of all protective groups may be performed in one or several steps by methods known to a person skilled in the art and described in the literature (e.g. T. W. Greene, P. G. M. Wuts, Protective groups in organic synthesis, 2^{nd} ed., 1991 John Wiley & Sons, Inc. New York).

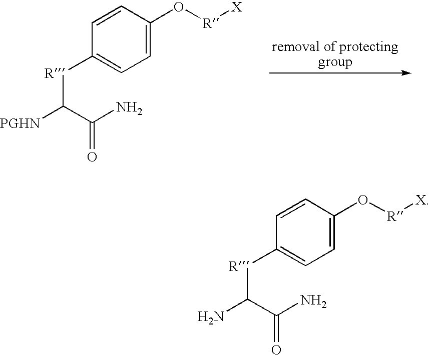

As defined in General Method (C)

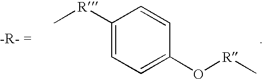

General Method (D):

A compound of the general formula

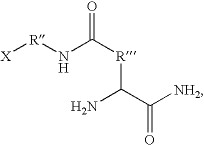

wherein R″ and R′″ are defined as above, may be prepared by a person skilled in the art, from a suitable acid, which is protected at the alpha-amino group by a suitable protecting group PG, known to a person skilled in the art and described in the literature e.g. in (e.g. T. W. Greene, P. G. M. Wuts, Protective groups in organic synthesis, 2^{nd} ed., 1991 John Wiley & Sons, Inc. New York)

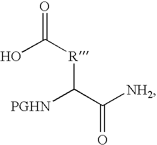

by reaction with a suitable primary or secondary amine, in which X may or may not be protected by a suitable protecting group, using acylation conditions known to a person skilled in the art e.g. a coupling reagent such as e.g. 1-hydroxybenzotriazole, 3,4-dihydro-3-hydroxybenzotriazin-4-one or 7-azabenzotriazole in combination with e.g. a carbodiimide such as e.g. diisopropylcarbodiimide or 1-(3-dimethylaminopropyl)-3-ethylcarbodiimide hydrochloride in the presence or absence of a suitable base such as e.g. triethylamine or ethyldiisopropylamine to form an amide

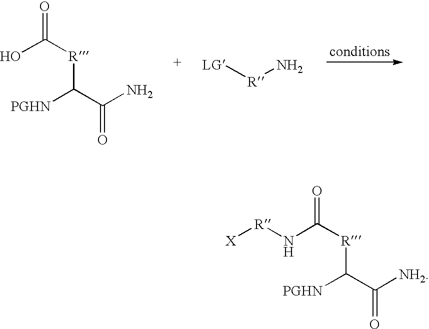

The removal of all protective groups may be performed in one or several steps by methods known to a person skilled in the art and described in the literature (e.g. T. W. Greene, P. G. M. Wuts, Protective groups in organic synthesis, 2^{nd} ed., 1991 John Wiley & Sons, Inc. New York).

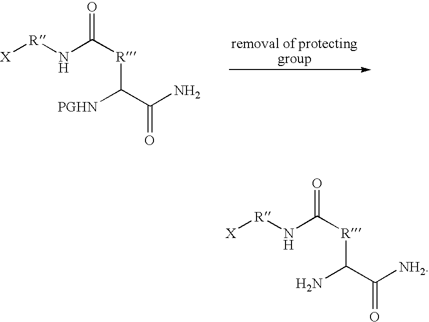

As defined in general Method (D)

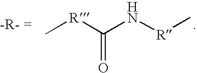

General Method (E): Synthesis of Ketogroup-Containing Amino Acid Amides from Cysteine

A conveniently N-protected cysteine derivative (for instance an ester, N-(2,4-dimethoxybenzyl)amide or N-bis(cyclopropyl)methyl amide) or conveniently N-protected cysteine amide is treated with a carbonyl-group-containing alkylating agent (R⁵⁰CO(CH₂)ₙLG″, LG″=leaving group for nucleophilic displacement selected from halogen, sulfonate (—O—SO₂—R⁵¹), dialkylsulfonium, phenyliodonium, or hydroxy, wherein R⁵¹ represents C₁₋₆alkyl, partially or completely fluorinated C₁₋₆alkyl, or aryl, optionally substituted with alkyl, halogen, nitro, cyano, or acetamido, and R⁵⁰ represents hydrogen, alkyl, aryl, or heteroaryl, said aryl or heteroaryl being optionally substituted once or several times with lower alkoxy, hydroxy, halogen, cyano, acyl, alkyl, or nitro, under suitable reaction conditions to yield an S-alkylated cysteine derivative. This derivative is converted into an amino acid amide by conversion of the acid derivative into an amide and deprotection of the alpha-amino group. Suitable N-protecting groups are for instance trityl, phthaloyl, or alkoxycarbonyl groups, such as tert-butyloxycarbonyl.

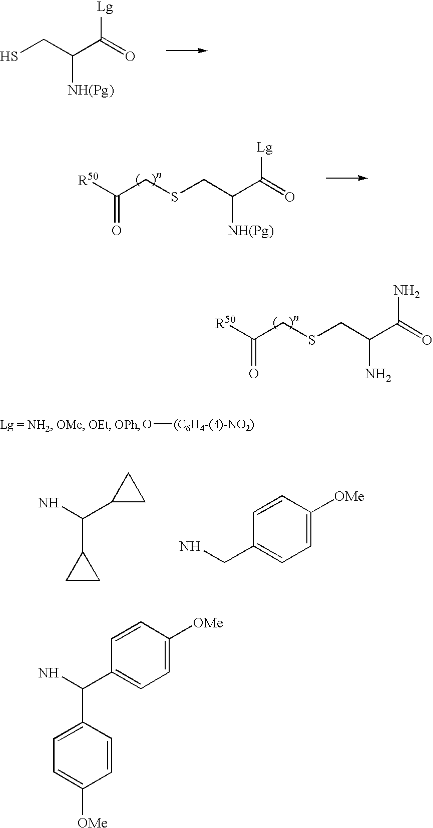

wherein n represents an integer from 1 to 10.

General Method (F): Synthesis of Ketogroup-Containing Amino Acid Amides from Aspartic or Glutamic Acid

Aspartic or glutamic acids can be selectively protected by treatment of an N-alkoxycarbonyl derivative with formaldehyde, to yield cyclic esters as shown below:

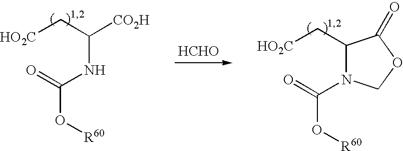

These derivatives, in which R⁶⁰ represents tert-butyl, benzyl, 2-chlorobenzyl, allyl, 2-(trimethylsilyl)ethyl, 2,2,2-trichloroethyl, or benzhydryl, can be converted to protected, ketone-containing amino acid derivatives by activation of the carboxylic acid (LvG representing halogen, aryloxy, or heteroaryloxy) and reaction with a carbon nucleophile R⁸⁰-M¹, in which R⁸⁰ represents alkyl, aryl, or heteroaryl, said aryl or heteroaryl being optionally substituted once or several times with lower alkoxy, hydroxy, halogen, cyano, acyl, alkyl, or nitro, and in which M¹ represents an alkali metal, Mg, Zn, Ti, Zr, Mn, Cu, Ce, or Ca, optionally in the presence of a suitable catalyst. Reaction of the product with ammonia and deprotection will yield the desired amino acid amide:

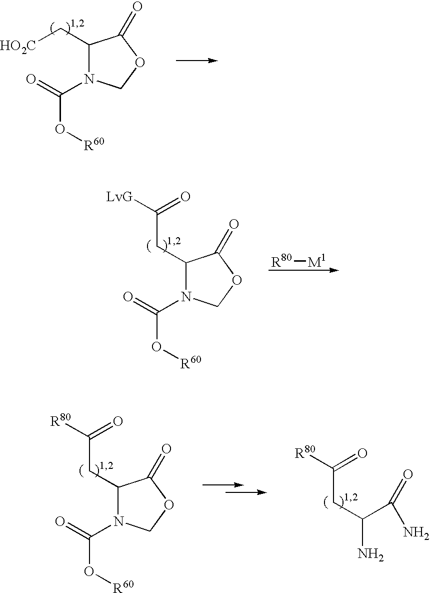

Similarly, reaction of N-alkoxycarbonyl pyroglutamic acid esters, in which R⁷⁰ represents tert-butyl, benzyl, 2-chlorobenzyl, allyl, 2-(trimethylsilyl)ethyl, 2,2,2-trichloroethyl, or benzhydryl, and R⁹⁰ represents lower alkyl, with nucleophilic carbon reagents can yield protected, keto-group-containing amino acid derivatives. Reaction of the product with ammonia and deprotection will yield the desired amino acid amide:

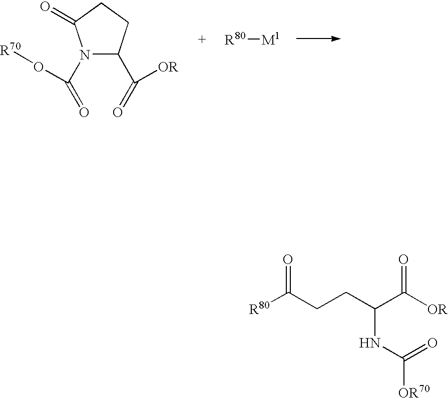

Similarly, suitably N-protected glutamic acid diesters as those shown below, in which R⁹⁰ represents lower alkyl, can be selectively acylated at carbon to yield, after hydrolysis and decarboxylation, protected derivatives of keto-group-containing amino acids, which can be converted into amino acid amides using standard procedures, well known to the skilled organic chemist.

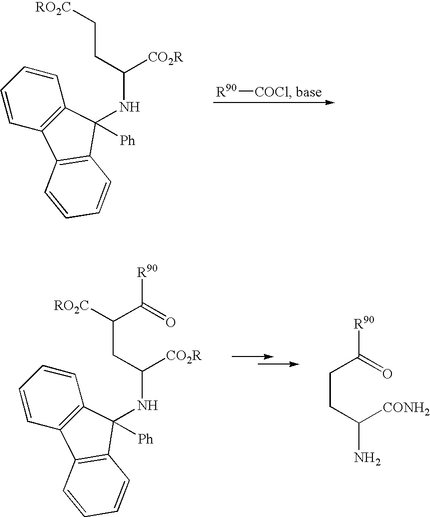

Similarly, reaction of suitably N-protected glutamic acid diesters as those shown below, in which R⁹⁰ represents lower alkyl, can be selectively acylated at carbon to yield, after hydrolysis and decarboxylation, protected derivatives of keto-group-containing amino acids, which can be converted into amino acid amides using standard procedures, well known to the skilled organic chemist.

The compound comprising the conjugating moiety, i.e. the compound of the formula Y-E-Z may either be acquired from commercial source, or it may be synthesized from readily available materials according to the following guidelines.

General Method (G)

A compound of the general formula

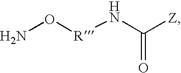

wherein R′″ represents C₁₋₁₅alkylene, C₂₋₁₅alkenylene, C₂₋₁₅alkynylene, C₁₋₁₅heteroalkylene, C₂₋₁₅heteroalkenylene, C₂₋₁₅heteroalkynylene, wherein one or more homocyclic aromatic compound biradical or heterocyclic compound biradical may be inserted, may be prepared from a suitable protected primary or secondary amine

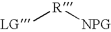

in which PG may be a suitable protection group, known to a person skilled in the art and described in the literature (e.g. T. W. Greene, P. G. M. Wuts, Protective groups in organic synthesis, 2^{nd} ed., 1991 John Wiley & Sons, Inc. New York, and wherein the anion of LG′″ is a leaving group, such as e.g. halogenide or sulfonate.

This amine is reacted with a suitable protected hydroxylamine

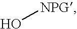

wherein PG′ is a protecting group, which is chosen in a way, that PG can be removed from an amine without removal of PG′ from the hydroxylamine. Examples for that can be found in the literature (e.g. T. W. Greene, P. G. M. Wuts, Protective groups in organic synthesis, 2^{nd} ed., 1991 John Wiley & Sons, Inc. New York).

The two components are reacted under basic conditions such as e.g. sodium hydride at a suitable temperature such as e.g −78° C. to 200° C.

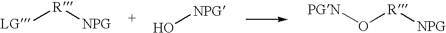

The protecting group of the amine may be removed selectively with a method described in the literature and known to a person skilled in the art

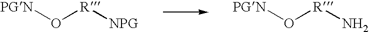

The amine may be acylated with a suitable acid and a coupling reagent such as e.g. 1-hydroxybenzotriazole, 3,4-dihydro-3-hydroxybenzotriazin-4-one or 7-azabenzotriazole in combination with e.g. a carbodiimide such as e.g. diisopropylcarbodiimide or 1-(3-dimethylaminopropyl)-3-ethylcarbodiimide hydrochloride in the presence or absence of a suitable base such as e.g. triethylamine or ethyldiisopropylamine to give an amide.

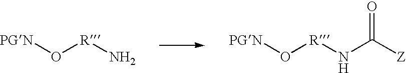

Finally, the protecting group of the hydroxylamine may be removed by a method described in the literature (e.g. T. W. Greene, P. G. M. Wuts, Protective groups in organic synthesis, 2^{nd} ed., 1991 John Wiley & Sons, Inc. New York) and known to a person skilled in the art to give the hydroxylamine.

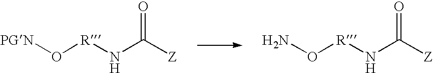

General Method (H)

A compound of the general formula

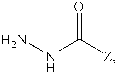

may be prepared from a suitable ester, in which R^{IV} is C₁₋₁₀alkyl in a suitable solvent such as ethanol by addition of hydrazine hydrate.

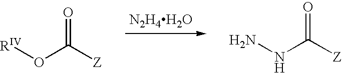

General Method (J) Transacylation Reaction

At a suitable temperature such as e.g. 5-50° C. or room temperature, a solution of the peptide in question (final concentration 1-10 mM) and the nucleophile in question (final concentration 10 mM-2M) is dissolved or suspended in water containing low concentrations of EDTA.

Organic solvents may be added to improve the solubility of the reactants. The mixture may be buffered to a suitable pH-value such as e.g. between pH 1 and pH 14, such as between pH 3.5 and pH 9, such as between pH 6 and pH 8.5, with a suitable buffer such as e.g. phosphate buffer, HEPES (2-[4-(2-hydroxyethyl)-1-piperazinyl)ethane sulfonic acid, or the pH can be maintained by addition of base or acid. A suitable enzyme is added to the said mixture of peptide and nucleophile. The reaction may be stopped after a suitable time e.g. between 5 min and 10 days, by changing temperature or pH-value, by adding organic solvents, or by dialysis or gel filtration.

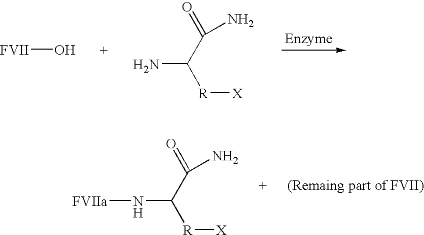

The pH of choice is determined e.g. by the solubility of the peptide to be conjugated and the activity of the enzyme to be used. Solubility of peptides is to a large extent determined by the pKa of the peptide. Normally, the solubility of a given peptide is at its minimum when pH equals pKa of the peptide. It lies within the skills of a skilled person to a pH at which to run the reaction taking due care to the above considerations.

General Method (K) Oxime Formation

An oxime moiety may be formed by dissolving the transacylated peptide in question, in which R^{V} may be a substituted or unsubstituted aromatic ring, a substituted or an unsubstituted heteroaromatic ring, hydrogen, or C₁₋₁₀alkyl, in water. Organic solvents may be added to increase solubility. The solution is buffered to a suitable pH-value such as e.g. between pH 0 and pH 14, between pH 3 and pH 6, or pH 5 and kept at a suitable temperature such as e.g. 0-60° C. The hydroxylamine in question is added, and oxime moiety is formed according to the reaction scheme below

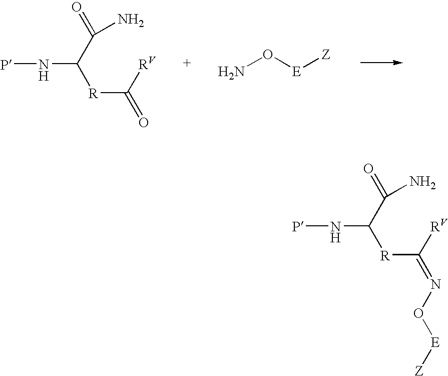

The pH of choice is determined e.g. by the solubility of the peptide to be. Solubility of peptides is to a large extent determined by the pKa of the peptide. Normally, the solubility of a given peptide is at its minimum when pH equals pKa of the peptide. It lies within the skills of a skilled person to a pH at which to run the reaction taking due care to the above consideration.

General Method (K) Hydrazone Formation

### Acylhydracone Formation (I)

An hydrazone moiety is formed by dissolving the transacylated peptide in question, in which R^{VI} may be a substituted or unsubstituted aromatic ring, a substituted or an unsubstituted heteroaromatic ring, hydrogen, or C₁₋₁₀alkyl, in water. The solution is buffered to a suitable pH-value such as e.g. between pH 2 and pH 14 or between pH 0 and pH 4 and kept at a suitable temperature such as e.g. 0-60° C. The hydrazide in question is added, whereby the hydrazone is formed

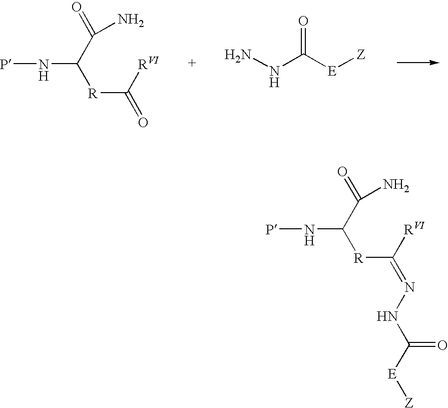

### Hydrazone Formation (II)

An hydrazone is formed by dissolving the transacylated peptide in question, in which R^{VII} may be a substituted or unsubstituted aromatic ring, a substituted or an unsubstituted heteroaromatic ring, hydrogen, or C₁₋₁₀alkyl, in water. The solution is buffered to a suitable pH-value such as e.g. between pH 2 and pH 14 or between pH 0 and pH 4 and kept at a suitable temperature such as e.g. 0-60° C. The hydrazine in question is added, whereby the hydrazone is formed

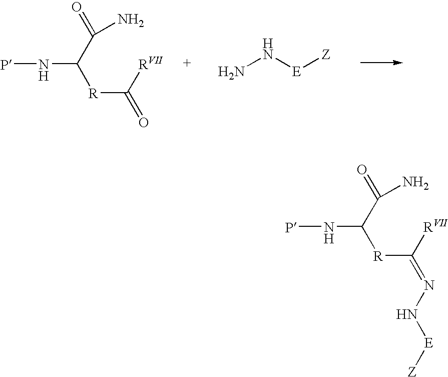

### Pharmaceutical Compositions

Another object of the present invention is to provide a pharmaceutical formulation comprising a compound according to the present invention which is present in a concentration from 0.0001 mg/ml to 1000 mg/ml, and wherein said formulation has a pH from 2.0 to 10.0. The formulation may further comprise a buffer system, preservative(s), tonicity agent(s), chelating agent(s), stabilizers and surfactants. In one embodiment of the invention the pharmaceutical formulation is an aqueous formulation, i.e. formulation comprising water. Such formulation is typically a solution or a suspension. In a further embodiment of the invention the pharmaceutical formulation is an aqueous solution. The term “aqueous formulation” is defined as a formulation comprising at least 50% w/w water. Likewise, the term “aqueous solution” is defined as a solution comprising at least 50% w/w water, and the term “aqueous suspension” is defined as a suspension comprising at least 50% w/w water.

In another embodiment the pharmaceutical formulation is a freeze-dried formulation, whereto the physician or the patient adds solvents and/or diluents prior to use.

In another embodiment the pharmaceutical formulation is a dried formulation (e.g. freeze-dried or spray-dried) ready for use without any prior dissolution.

In a further aspect the invention relates to a pharmaceutical formulation comprising an aqueous solution of the FVIIa-derivative, and a buffer, wherein said FVIIa-derivative is present in a concentration from 0.01 mg/ml or above, and wherein said formulation has a pH from about 2.0 to about 10.0.

In a another embodiment of the invention the pH of the formulation is selected from the list consisting of 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, and 10.0.

In a further embodiment of the invention the buffer is selected from the group consisting of sodium acetate, sodium carbonate, citrate, glycylglycine, histidine, glycine, lysine, arginine, sodium dihydrogen phosphate, disodium hydrogen phosphate, sodium phosphate, and tris(hydroxymethyl)-aminomethane, bicin, tricine, malic acid, succinate, maleic acid, fumaric acid, tartaric acid, aspartic acid or mixtures thereof. Each one of these specific buffers constitutes an alternative embodiment of the invention.

In a further embodiment of the invention the formulation further comprises a pharmaceutically acceptable preservative. In a further embodiment of the invention the preservative is selected from the group consisting of phenol, o-cresol, m-cresol, p-cresol, methyl p-hydroxybenzoate, propyl p-hydroxybenzoate, 2-phenoxyethanol, butyl p-hydroxybenzoate, 2-phenylethanol, benzyl alcohol, chlorobutanol, and thimerosal, bronopol, benzoic acid, imidurea, chlorohexidine, sodium dehydroacetate, chlorocresol, ethyl p-hydroxybenzoate, benzethonium chloride, chlorphenesin (3p-chlorphenoxypropane-1,2-diol) or mixtures thereof. In a further embodiment of the invention the preservative is present in a concentration from 0.1 mg/ml to 20 mg/ml. In a further embodiment of the invention the preservative is present in a concentration from 0.1 mg/ml to 5 mg/ml. In a further embodiment of the invention the preservative is present in a concentration from 5 mg/ml to 10 mg/ml. In a further embodiment of the invention the preservative is present in a concentration from 10 mg/ml to 20 mg/ml. Each one of these specific preservatives constitutes an alternative embodiment of the invention. The use of a preservative in pharmaceutical compositions is well-known to the skilled person. For convenience reference is made to Remington: The Science and Practice of Pharmacy, 19^{th} edition, 1995.

In a further embodiment of the invention the formulation further comprises an isotonic agent. In a further embodiment of the invention the isotonic agent is selected from the group consisting of a salt (e.g. sodium chloride), a sugar or sugar alcohol, an amino acid (e.g. L-glycine, L-histidine, arginine, lysine, isoleucine, aspartic acid, tryptophan, threonine),

an alditol (e.g. glycerol (glycerine), 1,2-propanediol (propyleneglycol), 1,3-propanediol, 1,3-butanediol) polyethyleneglycol (e.g. PEG400), or mixtures thereof. Any sugar such as mono-, di-, or polysaccharides, or water-soluble glucans, including for example fructose, glucose, mannose, sorbose, xylose, maltose, lactose, sucrose, trehalose, dextran, pullulan, dextrin, cyclodextrin, soluble starch, hydroxyethyl starch and carboxymethylcellulose-Na may be used. In one embodiment the sugar additive is sucrose. Sugar alcohol is defined as a C4-C8 hydrocarbon having at least one —OH group and includes, for example, mannitol, sorbitol, inositol, galactitol, dulcitol, xylitol, and arabitol. In one embodiment the sugar alcohol additive is mannitol. The sugars or sugar alcohols mentioned above may be used individually or in combination. There is no fixed limit to the amount used, as long as the sugar or sugar alcohol is soluble in the liquid preparation and does not adversely effect the stabilizing effects achieved using the methods of the invention. In one embodiment, the sugar or sugar alcohol concentration is between about 1 mg/ml and about 150 mg/ml. In a further embodiment of the invention the isotonic agent is present in a concentration from 1 mg/ml to 50 mg/ml. In a further embodiment of the invention the isotonic agent is present in a concentration from 1 mg/ml to 7 mg/ml. In a further embodiment of the invention the isotonic agent is present in a concentration from 8 mg/ml to 24 mg/ml. In a further embodiment of the invention the isotonic agent is present in a concentration from 25 mg/ml to 50 mg/ml. Each one of these specific isotonic agents constitutes an alternative embodiment of the invention. The use of an isotonic agent in pharmaceutical compositions is well-known to the skilled person. For convenience reference is made to Remington: The Science and Practice of Pharmacy, 19^{th} edition, 1995.

In a further embodiment of the invention the formulation further comprises a chelating agent. In a further embodiment of the invention the chelating agent is selected from salts of ethylenediaminetetraacetic acid (EDTA), citric acid, and aspartic acid, and mixtures thereof. In a further embodiment of the invention the chelating agent is present in a concentration from 0.1 mg/ml to 5 mg/ml. In a further embodiment of the invention the chelating agent is present in a concentration from 0.1 mg/ml to 2 mg/ml. In a further embodiment of the invention the chelating agent is present in a concentration from 2 mg/ml to 5 mg/ml. Each one of these specific chelating agents constitutes an alternative embodiment of the invention. The use of a chelating agent in pharmaceutical compositions is well-known to the skilled person. For convenience reference is made to Remington: The Science and Practice of Pharmacy, 19^{th} edition, 1995.

In a further embodiment of the invention the formulation further comprises a stabilizer. The use of a stabilizer in pharmaceutical compositions is well-known to the skilled person. For convenience reference is made to Remington: The Science and Practice of Pharmacy, 19^{th} edition, 1995.

More particularly, compositions of the invention are stabilized liquid pharmaceutical compositions whose therapeutically active components include a polypeptide that possibly exhibits aggregate formation during storage in liquid pharmaceutical formulations. By “aggregate formation” is intended a physical interaction between the polypeptide molecules that results in formation of oligomers, which may remain soluble, or large visible aggregates that precipitate from the solution. By “during storage” is intended a liquid pharmaceutical composition or formulation once prepared, is not immediately administered to a subject. Rather, following preparation, it is packaged for storage, either in a liquid form, in a frozen state, or in a dried form for later reconstitution into a liquid form or other form suitable for administration to a subject. By “dried form” is intended the liquid pharmaceutical composition or formulation is dried either by freeze drying (i.e., lyophilization; see, for example, Williams and Polli (1984) J. Parenteral Sci. Technol. 38:48-59), spray drying (see Masters (1991) in Spray-Drying Handbook (5th ed; Longman Scientific and Technical, Essez, U.K.), pp. 491-676; Broadhead et al. (1992) Drug Devel. Ind. Pharm. 18:1169-1206; and Mumenthaler et al. (1994) Pharm. Res. 11:12-20), or air drying (Carpenter and Crowe (1988) Cryobiology 25:459-470; and Roser (1991) Biopharm. 4:47-53). Aggregate formation by a polypeptide during storage of a liquid pharmaceutical composition can adversely affect biological activity of that polypeptide, resulting in loss of therapeutic efficacy of the pharmaceutical composition. Furthermore, aggregate formation may cause other problems such as blockage of tubing, membranes, or pumps when the polypeptide-containing pharmaceutical composition is administered using an infusion system.

The pharmaceutical compositions of the invention may further comprise an amount of an amino acid base sufficient to decrease aggregate formation by the polypeptide during storage of the composition. By “amino acid base” is intended an amino acid or a combination of amino acids, where any given amino acid is present either in its free base form or in its salt form. Where a combination of amino acids is used, all of the amino acids may be present in their free base forms, all may be present in their salt forms, or some may be present in their free base forms while others are present in their salt forms. In one embodiment, amino acids to use in preparing the compositions of the invention are those carrying a charged side chain, such as arginine, lysine, aspartic acid, and glutamic acid. Any stereoisomer (i.e., L, D, or DL isomer) of a particular amino acid (e.g. glycine, methionine, histidine, imidazole, arginine, lysine, isoleucine, aspartic acid, tryptophan, threonine and mixtures thereof) or combinations of these stereoisomers, may be present in the pharmaceutical compositions of the invention so long as the particular amino acid is present either in its free base form or its salt form. In one embodiment the L-stereoisomer is used. Compositions of the invention may also be formulated with analogues of these amino acids. By “amino acid analogue” is intended a derivative of the naturally occurring amino acid that brings about the desired effect of decreasing aggregate formation by the polypeptide during storage of the liquid pharmaceutical compositions of the invention. Suitable arginine analogues include, for example, aminoguanidine, ornithine and N-monoethyl L-arginine, suitable methionine analogues include ethionine and buthionine and suitable cysteine analogues include S-methyl-L cysteine. As with the other amino acids, the amino acid analogues are incorporated into the compositions in either their free base form or their salt form. In a further embodiment of the invention the amino acids or amino acid analogues are used in a concentration, which is sufficient to prevent or delay aggregation of the protein.

In a further embodiment of the invention methionine (or other sulphuric amino acids or amino acid analogous) may be added to inhibit oxidation of methionine residues to methionine sulfoxide when the polypeptide acting as the therapeutic agent is a polypeptide comprising at least one methionine residue susceptible to such oxidation. By “inhibit” is intended minimal accumulation of methionine oxidized species over time. Inhibiting methionine oxidation results in greater retention of the polypeptide in its proper molecular form. Any stereoisomer of methionine (L, D, or DL isomer) or combinations thereof can be used. The amount to be added should be an amount sufficient to inhibit oxidation of the methionine residues such that the amount of methionine sulfoxide is acceptable to regulatory agencies. Typically, this means that the composition contains no more than about 10% to about 30% methionine sulfoxide. Generally, this can be achieved by adding methionine such that the ratio of methionine added to methionine residues ranges from about 1:1 to about 1000:1, such as 10:1 to about 100:1.

In a further embodiment of the invention the formulation further comprises a stabilizer selected from the group of high molecular weight polymers or low molecular compounds. In a further embodiment of the invention the stabilizer is selected from polyethylene glycol (e.g. PEG 3350), polyvinyl alcohol (PVA), polyvinylpyrrolidone, carboxy-/hydroxycellulose or derivates thereof (e.g. HPC, HPC-SL, HPC-L and HPMC), cyclodextrins, sulphur-containing substances as monothioglycerol, thioglycolic acid and 2-methylthioethanol, and different salts (e.g. sodium chloride). Each one of these specific stabilizers constitutes an alternative embodiment of the invention.

The pharmaceutical compositions may also comprise additional stabilizing agents, which further enhance stability of a therapeutically active polypeptide therein. Stabilizing agents of particular interest to the present invention include, but are not limited to, methionine and EDTA, which protect the polypeptide against methionine oxidation, and a nonionic surfactant, which protects the polypeptide against aggregation associated with freeze-thawing or mechanical shearing.

In a further embodiment of the invention the formulation further comprises a surfactant. In a further embodiment of the invention the surfactant is selected from a detergent, ethoxylated castor oil, polyglycolized glycerides, acetylated monoglycerides, sorbitan fatty acid esters, polyoxypropylene-polyoxyethylene block polymers (eg. poloxamers such as Pluronic® F68, poloxamer 188 and 407, Triton X-100), polyoxyethylene sorbitan fatty acid esters, polyoxyethylene and polyethylene derivatives such as alkylated and alkoxylated derivatives (tweens, e.g. Tween-20, Tween-40, Tween-80 and Brij-35), monoglycerides or ethoxylated derivatives thereof, diglycerides or polyoxyethylene derivatives thereof, alcohols, glycerol, lectins and phospholipids (eg. phosphatidyl serine, phosphatidyl choline, phosphatidyl ethanolamine, phosphatidyl inositol, diphosphatidyl glycerol and sphingomyelin), derivates of phospholipids (eg. dipalmitoyl phosphatidic acid) and lysophospholipids (eg. palmitoyl lysophosphatidyl-L-serine and 1-acyl-sn-glycero-3-phosphate esters of ethanolamine, choline, serine or threonine) and alkyl, alkoxyl (alkyl ester), alkoxy (alkyl ether)-derivatives of lysophosphatidyl and phosphatidylcholines, e.g. lauroyl and myristoyl derivatives of lysophosphatidylcholine, dipalmitoylphosphatidylcholine, and modifications of the polar head group, that is cholines, ethanolamines, phosphatidic acid, serines, threonines, glycerol, inositol, and the positively charged DODAC, DOTMA, DCP, BISHOP, lysophosphatidylserine and lysophosphatidylthreonine, and glycerophospholipids (eg. cephalins), glyceroglycolipids (eg. galactopyranoside), sphingoglycolipids (eg. ceramides, gangliosides), dodecylphosphocholine, hen egg lysolecithin, fusidic acid derivatives—(e.g. sodium tauro-dihydrofusidate etc.), long-chain fatty acids and salts thereof C6-C12 (eg. oleic acid and caprylic acid), acylcarnitines and derivatives, N″-acylated derivatives of lysine, arginine or histidine, or side-chain acylated derivatives of lysine or arginine, N″-acylated derivatives of dipeptides comprising any combination of lysine, arginine or histidine and a neutral or acidic amino acid, N″-acylated derivative of a tripeptide comprising any combination of a neutral amino acid and two charged amino acids, DSS (docusate sodium, CAS registry no [577-11-7]), docusate calcium, CAS registry no [128-49-4]), docusate potassium, CAS registry no [7491-09-0]), SDS (sodium dodecyl sulphate or sodium lauryl sulphate), sodium caprylate, cholic acid or derivatives thereof, bile acids and salts thereof and glycine or taurine conjugates, ursodeoxycholic acid, sodium cholate, sodium deoxycholate, sodium taurocholate, sodium glycocholate, N-Hexadecyl-N,N-dimethyl-3-ammonia-1-propanesulfonate, anionic (alkyl-aryl-sulphonates) monovalent surfactants, zwitterionic surfactants (e.g. N-alkyl-N,N-dimethylammonio-1-propanesulfonates, 3-cholamido-1-propyldimethylammonio-1-propanesulfonate, cationic surfactants (quaternary ammonium bases) (e.g. cetyl-trimethylammonium bromide, cetylpyridinium chloride), non-ionic surfactants (eg. Dodecyl β-D-glucopyranoside), poloxamines (eg. Tetronic's), which are tetrafunctional block copolymers derived from sequential addition of propylene oxide and ethylene oxide to ethylenediamine, or the surfactant may be selected from the group of imidazoline derivatives, or mixtures thereof. Each one of these specific surfactants constitutes an alternative embodiment of the invention.

The use of a surfactant in pharmaceutical compositions is well-known to the skilled person. For convenience reference is made to Remington: The Science and Practice of Pharmacy, 19^{th} edition, 1995.

It is possible that other ingredients may be present in the peptide pharmaceutical formulation of the present invention. Such additional ingredients may include wetting agents, emulsifiers, antioxidants, bulking agents, tonicity modifiers, chelating agents, metal ions, oleaginous vehicles, proteins (e.g., human serum albumin, gelatine or proteins) and a zwitterion (e.g., an amino acid such as betaine, taurine, arginine, glycine, lysine and histidine). Such additional ingredients, of course, should not adversely affect the overall stability of the pharmaceutical formulation of the present invention.

Pharmaceutical compositions containing a FVIIa-derivative according to the present invention may be administered to a patient in need of such treatment at several sites, for example, at topical sites, for example, skin and mucosal sites, at sites which bypass absorption, for example, administration in an artery, in a vein, in the heart, and at sites which involve absorption, for example, administration in the skin, under the skin, in a muscle or in the abdomen.

Administration of pharmaceutical compositions according to the invention may be through several routes of administration, for example, lingual, sublingual, buccal, in the mouth, oral, in the stomach and intestine, nasal, pulmonary, for example, through the bronchioles and alveoli or a combination thereof, epidermal, dermal, transdermal, vaginal, rectal, ocular, for examples through the conjunctiva, uretal, and parenteral to patients in need of such a treatment.

Compositions of the current invention may be administered in several dosage forms, for example, as solutions, suspensions, emulsions, microemulsions, multiple emulsion, foams, salves, pastes, plasters, ointments, tablets, coated tablets, rinses, capsules, for example, hard gelatine capsules and soft gelatine capsules, suppositories, rectal capsules, drops, gels, sprays, powder, aerosols, inhalants, eye drops, ophthalmic ointments, ophthalmic rinses, vaginal pessaries, vaginal rings, vaginal ointments, injection solution, in situ transforming solutions, for example in situ gelling, in situ setting, in situ precipitating, in situ crystallization, infusion solution, and implants.

Compositions of the invention may further be compounded in, or attached to, for example through covalent, hydrophobic and electrostatic interactions, a drug carrier, drug delivery system and advanced drug delivery system in order to further enhance stability of the FVIIa-derivative, increase bioavailability, increase solubility, decrease adverse effects, achieve chronotherapy well known to those skilled in the art, and increase patient compliance or any combination thereof. Examples of carriers, drug delivery systems and advanced drug delivery systems include, but are not limited to, polymers, for example cellulose and derivatives, polysaccharides, for example dextran and derivatives, starch and derivatives, poly(vinyl alcohol), acrylate and methacrylate polymers, polylactic and polyglycolic acid and block co-polymers thereof, polyethylene glycols, carrier proteins, for example albumin, gels, for example, thermogelling systems, for example block co-polymeric systems well known to those skilled in the art, micelles, liposomes, microspheres, nanoparticulates, liquid crystals and dispersions thereof, L2 phase and dispersions there of, well known to those skilled in the art of phase behaviour in lipid-water systems, polymeric micelles, multiple emulsions, self-emulsifying, self-microemulsifying, cyclodextrins and derivatives thereof, and dendrimers.

Compositions of the current invention are useful in the formulation of solids, semisolids, powder and solutions for pulmonary administration of the compound, using, for example a metered dose inhaler, dry powder inhaler and a nebulizer, all being devices well known to those skilled in the art.

Compositions of the current invention are specifically useful in the formulation of controlled, sustained, protracting, retarded, and slow release drug delivery systems. More specifically, but not limited to, compositions are useful in formulation of parenteral controlled release and sustained release systems (both systems leading to a many-fold reduction in number of administrations), well known to those skilled in the art. Even more preferably, are controlled release and sustained release systems administered subcutaneous. Without limiting the scope of the invention, examples of useful controlled release system and compositions are hydrogels, oleaginous gels, liquid crystals, polymeric micelles, microspheres, nanoparticles,

Methods to produce controlled release systems useful for compositions of the current invention include, but are not limited to, crystallization, condensation, co-crystallization, precipitation, co-precipitation, emulsification, dispersion, high pressure homogenisation, encapsulation, spray drying, microencapsulating, coacervation, phase separation, solvent evaporation to produce microspheres, extrusion and supercritical fluid processes. General reference is made to Handbook of Pharmaceutical Controlled Release (Wise, D. L., ed. Marcel Dekker, New York, 2000) and Drug and the Pharmaceutical Sciences vol. 99: Protein Formulation and Delivery (MacNally, E. J., ed. Marcel Dekker, New York, 2000).

Parenteral administration may be performed by subcutaneous, intramuscular, intraperitoneal or intravenous injection by means of a syringe, optionally a pen-like syringe. Alternatively, parenteral administration can be performed by means of an infusion pump. A further option is a composition which may be a solution or suspension for the administration of the [the protein] compound in the form of a nasal or pulmonal spray. As a still further option, the pharmaceutical compositions containing the [the protein] compound of the invention can also be adapted to transdermal administration, e.g. by needle-free injection or from a patch, optionally an iontophoretic patch, or transmucosal, e.g. buccal, administration.

The term “stabilized formulation” refers to a formulation with increased physical stability, increased chemical stability or increased physical and chemical stability.

The term “physical stability” of the protein formulation as used herein refers to the tendency of the protein to form biologically inactive and/or insoluble aggregates of the protein as a result of exposure of the protein to thermo-mechanical stresses and/or interaction with interfaces and surfaces that are destabilizing, such as hydrophobic surfaces and interfaces. Physical stability of the aqueous protein formulations is evaluated by means of visual inspection and/or turbidity measurements after exposing the formulation filled in suitable containers (e.g. cartridges or vials) to mechanical/physical stress (e.g. agitation) at different temperatures for various time periods. Visual inspection of the formulations is performed in a sharp focused light with a dark background. The turbidity of the formulation is characterized by a visual score ranking the degree of turbidity for instance on a scale from 0 to 3 (a formulation showing no turbidity corresponds to a visual score 0, and a formulation showing visual turbidity in daylight corresponds to visual score 3). A formulation is classified physical unstable with respect to protein aggregation, when it shows visual turbidity in daylight. Alternatively, the turbidity of the formulation can be evaluated by simple turbidity measurements well-known to the skilled person. Physical stability of the aqueous protein formulations can also be evaluated by using a spectroscopic agent or probe of the conformational status of the protein. The probe is preferably a small molecule that preferentially binds to a non-native conformer of the protein. One example of a small molecular spectroscopic probe of protein structure is Thioflavin T. Thioflavin T is a fluorescent dye that has been widely used for the detection of amyloid fibrils. In the presence of fibrils, and perhaps other protein configurations as well, Thioflavin T gives rise to a new excitation maximum at about 450 nm and enhanced emission at about 482 nm when bound to a fibril protein form. Unbound Thioflavin T is essentially non-fluorescent at the wavelengths.

Other small molecules can be used as probes of the changes in protein structure from native to non-native states. For instance the “hydrophobic patch” probes that bind preferentially to exposed hydrophobic patches of a protein. The hydrophobic patches are generally buried within the tertiary structure of a protein in its native state, but become exposed as a protein begins to unfold or denature. Examples of these small molecular, spectroscopic probes are aromatic, hydrophobic dyes, such as anthracene, acridine, phenanthroline or the like. Other spectroscopic probes are metal-amino acid complexes, such as cobalt metal complexes of hydrophobic amino acids, such as phenylalanine, leucine, isoleucine, methionine, and valine, or the like.

The term “chemical stability” of the protein formulation as used herein refers to chemical covalent changes in the protein structure leading to formation of chemical degradation products with potential less biological potency and/or potential increased immunogenic properties compared to the native protein structure. Various chemical degradation products can be formed depending on the type and nature of the native protein and the environment to which the protein is exposed. Elimination of chemical degradation can most probably not be completely avoided and increasing amounts of chemical degradation products is often seen during storage and use of the protein formulation as well-known by the person skilled in the art. Most proteins are prone to deamidation, a process in which the side chain amide group in glutaminyl or asparaginyl residues is hydrolysed to form a free carboxylic acid. Other degradations pathways involves formation of high molecular weight transformation products where two or more protein molecules are covalently bound to each other through transamidation and/or disulfide interactions leading to formation of covalently bound dimer, oligomer and polymer degradation products (Stability of Protein Pharmaceuticals, Ahern. T. J. & Manning M. C., Plenum Press, New York 1992). Oxidation (of for instance methionine residues) can be mentioned as another variant of chemical degradation. The chemical stability of the protein formulation can be evaluated by measuring the amount of the chemical degradation products at various time-points after exposure to different environmental conditions (the formation of degradation products can often be accelerated by for instance increasing temperature). The amount of each individual degradation product is often determined by separation of the degradation products depending on molecule size and/or charge using various chromatography techniques (e.g. SEC-HPLC and/or RP-HPLC).

Hence, as outlined above, a “stabilized formulation” refers to a formulation with increased physical stability, increased chemical stability or increased physical and chemical stability. In general, a formulation must be stable during use and storage (in compliance with recommended use and storage conditions) until the expiration date is reached.

In one embodiment of the invention the pharmaceutical formulation comprising the compound is stable for more than 6 weeks of usage and for more than 3 years of storage.

In another embodiment of the invention the pharmaceutical formulation comprising the compound is stable for more than 4 weeks of usage and for more than 3 years of storage.

In a further embodiment of the invention the pharmaceutical formulation comprising the compound is stable for more than 4 weeks of usage and for more than two years of storage.

In an even further embodiment of the invention the pharmaceutical formulation comprising the compound is stable for more than 2 weeks of usage and for more than two years of storage.

### EXAMPLES

### Example 1

### Auto-Catalytic Transacylation Mediated by FVIIa

The auto-catalytic transacylation of FVIIa light chain starts using purified or semi-purified FVII zymogen at 10-25 uM in a suitable buffer not containing primary amines which may interfere with the reaction, e.g., 20 mM HEPES, 100 mM NaCl, 10 mM CaCl₂, pH 8.0 or 200 mM Na₂CO₃, 10 mM CaCl₂, pH 9.5, to this solution is added L-Phe(4-COCH₃)—NH₂ to a final concentration of 100 mM and the reaction mixture is placed at 25° C. Samples are withdrawn at various time points and the progress is monitored by RP-HPLC following reduction by TCEP to separate light from heavy chain and thus, facilitate interpretation of the mixture. Once >80% of the zymogen has been converted to FVIIa (typically 48-72 hours) the reaction is stopped by addition of 20 mM EDTA and the complete mixture is captured on a Q-sepharose column which is washed with 10 column volumes of 20 mM Tris, 150 mM NaCl, pH 8.0 and the bound material is eluted with a 10 column volume 0-50 mM CaCl₂ gradient in the same buffer.

To the released material is then added 50 fold excess of H₂N—O-PEG(20000) and the reaction mixture is incubated at room temperature for 16 hours. Upon completion of the reaction, the material is again captured on Q-sepharose as described above in order to remove unreacted PEG, but is this time eluted directly onto a Superdex 200 gelfiltration column with 20 mM Tris, 150 mM NaCl, 20 mM CaCl₂, pH 8.0. The modified and non-modified material will separate significantly as the modified has an apparent mass of 3-4 times that of the non-modified. The modified material may then be characterized for FVIIa activity, Tissue factor binding, FX activation activity and the ability to induce clot formation in a variety of assays all known to those skilled in the art. Furthermore, the material may be characterized in PK models.

### Example 2

### Transacylation Mediated by FVII Activating Protease

The transacylation of FVIIa light chain mediated by FSAP (FVII activating protease) is essentially an accelerated version of Example 1 as FVII auto-activates rather slowly. Again the process starts using purified or semi-purified FVII zymogen at 10-25 uM in a suitable buffer not containing primary amines which may interfere with the reaction, e.g., 20 mM HEPES, 100 mM NaCl, 10 mM CaCl₂, pH 8.0 or 200 mM Na₂CO₃, 10 mM CaCl₂, pH 9.5, to this solution is added L-Phe(4-COCH₃)—NH₂ and FSAP to a final concentration of 100 mM and 50 nM, respectively and the reaction mixture is incubated at 25° C. Samples are withdrawn at various time points and the progress is monitored by RP-HPLC following reduction by TCEP to separate light from heavy chain and thus, facilitate interpretation of the mixture. Once >80% of the zymogen has been converted to FVIIa (typically >24 hours) the reaction is stopped by addition of 20 mM EDTA and the complete mixture is captured on a Q-sepharose column which is washed with 10 column volumes of 20 mM Tris, 150 mM NaCl, pH 8.0 and the bound material is eluted with a 10 column volume 0-50 mM CaCl₂ gradient in the same buffer.

To the released material is then added 50 fold excess of H₂N—O-PEG(20000) and the reaction mixture is incubated at room temperature for 16 hours. Upon completion of the reaction, the material is again captured on Q-sepharose as described above in order to remove unreacted PEG, but is this time eluted directly onto a Superdex 200 gelfiltration column with 20 mM Tris, 150 mM NaCl, 20 mM CaCl₂, pH 8.0. The modified and non-modified material will separate significantly as the modified has an apparent mass of 3-4 times that of the non-modified. The modified material may then be characterized for FVIIa activity, Tissue factor binding, FX activation activity and the ability to induce clot formation in a variety of assays all known to those skilled in the art. Furthermore, the material may be characterized in PK models.

### Example 3

### Transacylation Mediated by Sortase A or B to Introduce a Specific Modification Site

This approach requires the introduction of a modified activation site in FVIIa. Thus, using state of the art molecular biology methods, e.g. overlap PCR (Higuchi, 1989) or QuickChange™ (Invitrogen, Inc.) the endogenous FVIIa activation site KPQGR¹⁵²-I¹⁵³VGG may be changed to a Sortase A recognition site (LPQTG¹⁵²-I¹⁵³VGG) or a Sortase B recognition site (NPQTN¹⁵²-I¹⁵³VGG) using the oligo nucleotides pairs:

```
1. F7 SrtA forw: 5′-AAAAGAAATGCCAGCCTACCCCAAACCGGTATTGTGGGGGGCAAG-3′
      F7 SrtA reverse: 5′-CTTGCCCCCCACAATACCGGTTTGGGGTAGGCTGGCATTTCTTTT-3′
2. F7 SrtB forw: 5′-AAAAGAAATGCCAGCAATCCCCAAACCAATATTGTGGGGGGCAAG-3′
      F7 SrtB reverse: 5′-CTTGCCCCCCACAATATTGGTTTGGGGATTGCTGGCATTTCTTTT-3′
```

The resulting products was cloned into the pIRES expression vector and verified by dye-deoxy DNA sequencing using an ABI DNA sequencer. FVIIa was then expressed and purified as previously described (ref.).

The generation starts using purified or semi-purified FVII (SrtA) or FVII (SrtB) zymogen at 10-25 uM in a buffer not containing primary amines which may interfere with the reaction, i.e., 20 mM Tris, 150 mM NaCl, 10 mM CaCl₂, pH 8.0. To this solution is added Gly₄-HN—CH₂—CH₂-O—NH₂ to a final concentration of 5 mM and SrtA or B (1 μM final concentration) depending on the zymogen used and the mixture is incubated at 25° C. until >80% of the zymogen has been converted to FVIIa as judged by reducing RP-HPLC (typically >24 hours). At which time EDTA is added to a final concentration of 20 mM and the complete mixture is captured on a Q-sepharose column which is washed with 10 column volumes of 20 mM Tris, 150 mM NaCl, pH 8.0 and the bound material is eluted with a 10 column volume 0-50 mM CaCl₂ gradient in the same buffer.

To the released material is then added 50 fold excess of PEG(20000)-aldehyde and the reaction mixture is incubated at room temperature for 16 hours. Upon completion of the reaction, the material is again captured on Q-sepharose as described above in order to remove unreacted PEG, but is this time eluted directly onto a Superdex 200 gelfiltration column with 20 mM Tris, 150 mM NaCl, 20 mM CaCl₂, pH 8.0. The modified and non-modified material will separate significantly as the modified has an apparent mass of 3-4 times that of the non-modified. The modified material may then be characterized for FVIIa activity, Tissue factor binding, FX activation activity and the ability to induce clot formation in a variety of assays all known to those skilled in the art. Furthermore, the material may be characterized in PK models.

Higuchi, R. (1989) In: Erhlich H A, eds. PCR technology: principles and applications for DNA amplification. New York: Stockton, pp. 61-70.

### Example 4

### Transacylation Mediated by Sortase A or B to Introduce PEG 20000

The generation starts using purified or semi-purified FVII (SrtA) or FVII (SrtB) zymogen at 10-25 μM (prepared as described above) in a buffer not containing primary amines which may interfere with the reaction, i.e., 20 mM Tris, 150 mM NaCl, 10 mM CaCl₂, pH 8.0. To this solution is added Gly₅-PEG20000 to a final concentration of 5 mM and SrtA or B (1 μM final concentration) depending on the zymogen used and the mixture is incubated at 25° C. until >80% of the zymogen has been converted to FVIIa as judged by reducing RP-HPLC (typically >24 hours). At which time EDTA is added to a final concentration of 20 mM and the complete mixture is captured on a Q-sepharose column which is washed with 10 column volumes of 20 mM Tris, 150 mM NaCl, pH 8.0 and the bound material is eluted directly onto a Superdex 200 gelfiltration column with 20 mM Tris, 150 mM NaCl, 20 mM CaCl₂, pH 8.0. The modified and non-modified material will separate significantly as the modified has an apparent mass of 3-4 times that of the non-modified. The modified material may then be characterized for FVIIa activity, Tissue factor binding, FX activation activity and the ability to induce clot formation in a variety of assays all known to those skilled in the art. Furthermore, the material may be characterized in PK models.

All references, including publications, patent applications, and patents, cited herein are hereby incorporated by reference in their entirety and to the same extent as if each reference were individually and specifically indicated to be incorporated by reference and were set forth in its entirety herein (to the maximum extent permitted by law), regardless of any separately provided incorporation of particular documents made elsewhere herein.

The use of the terms “a” and “an” and “the” and similar referents in the context of describing the invention are to be construed to cover both the singular and the plural, unless otherwise indicated herein or clearly contradicted by context.

Unless otherwise stated, all exact values provided herein are representative of corresponding approximate values (e.g., all exact exemplary values provided with respect to a particular factor or measurement can be considered to also provide a corresponding approximate measurement, modified by “about,” where appropriate).

The description herein of any aspect or aspect of the invention using terms such as “comprising”, “having,” “including,” or “containing” with reference to an element or elements is intended to provide support for a similar aspect or aspect of the invention that “consists of”, “consists essentially of”, or “substantially comprises” that particular element or elements, unless otherwise stated or clearly contradicted by context (e.g., a composition described herein as comprising a particular element should be understood as also describing a composition consisting of that element, unless otherwise stated or clearly contradicted by context).

Exemplary embodiments and features of the invention include, without limitation, those provided in the following list:

1. A method of obtaining a derivate of FVIIa, P′—R—X, comprising the step of cleaving FVII or a FVII variant enzymatically in the presence of R′—X to conjugate —R—X at the enzymatically generated C-terminal of FVIIa:

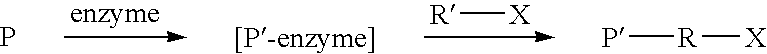

wherein P represents FVII or a FVII variant, P′ represents the product of the cleavage, R′—X represents the compound reacting with P′, X represents the group to be conjugated to P′ or X represents a functional group, R′ represents R comprising a nucleophilic group.

2. The method of embodiment 1, wherein the obtained product P′—R—X wherein X represents a functional group, is further reacted with a compound of the general formula Y-E-Z to obtain a product

P′—R-A-E-Z

wherein R represents a linker or a bond;
wherein P′ represents the FVII polypeptide product of the enzymatically cleavage of FVII;

X represents a radical comprising a functional group capable of reacting with Y;

Y represents a radical comprising one or more functional groups which groups are capable of reacting with X;

E represents a linker or a bond;

A represents the moiety formed by the reaction between the functional groups comprised in X and Y; and

Z is the moiety to be conjugated to the peptide.

3. The method according to any of embodiment 1 or 2, wherein FVII is cleaved by FVIIa it self, FIXa, FSAP, Hepsin, and matriptase.

4. The method of embodiment 2, wherein X and Y are selected from amongst carbonyl groups, such as keto and aldehyde groups, and amino derivatives, such as an amino acid, NH—NH₂, —NH—NH₂, —O—C(O)—NH—NH₂, —NH—C(O)—NH—NH₂, NH—C(S)—NH—NH₂, —NHC(O)—NH—NH—C(O)—NH—NH₂, NH—NH—C(O)—NH—NH₂, —NH—NH—C(S)—NH—NH₂, —NH—C(O)—C₆H₄—NH—NH₂, C(O)—NH—NH₂, —O—NH₂, —C(O)—O—NH₂, —NH—C(O)—O—NH₂ and —NH—C(S)—O—NH₂.

5. The method of embodiment 4, wherein Y is an amino acid, or a derivative of —NH—NH₂, —O—C(O)—NH—NH₂, NH—C(O)—NH—NH₂, —NH—C(S)—NH—NH₂, NHC(O)—NH—NH—C(O)—NH—NH₂, —NH—NH—C(O)—NH—NH₂, NH—NH—C(S)—NH—NH₂, —NH—C(O)—C₆H₄—NH—NH₂, —C(O)—NH—NH₂, —O—NH₂, —C(O)—O—NH₂, —NH—C(O)—O—NH₂ and —NH—C(S)—O—NH₂ and X is a keto- or an aldehyde-functionality.

6. The method of embodiments 1, wherein R′—X comprises an amino acid or a number of amino acids wherein one of the amino acids are derivatised to include further functional groups for derivatising or the amino acid contains the group Z.

7. The method of embodiment 5, wherein R′—X is an α-amino acid derivative

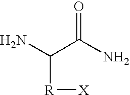

wherein R is a suitable linker and X is as defined in embodiment 1.

8. The method of embodiment 5, wherein R′—X is selected from the group consisting of G₍₁₋₅₎-PEG, G₍₁₋₅₎-lipid, G₍₁₋₄₎-NH—CH₂—CHO, and G₍₁₋₄₎-NH—(CH₂)ₙ—O—NH₂, wherein n is ≧2, such as 2.

9. The method according to any one of embodiments 1-8, wherein Z is PEG or C₅-C₂₄ fatty acid, aliphatic C₅-C₂₄diacid.

10. Isolated FVII polypeptides having the sequence of SEQ ID NO. 1 or SEQ ID NO 2.

11. A FVIIa derivative P′—R—X, wherein P′ represents a FVII polypeptide product of an enzymatic cleavage of FVII; X represents the group to be conjugated to P′ or X represents a functional group; R represent a linker or a bond to the enzymatically generated C-terminal of FVIIa.

12. A FVIIa derivative P′—R-A-E-Z, wherein P′ represents the FVII polypeptide product of an enzymatic cleavage of FVII; E represents a linker or a bond; A represents a chemical moiety; R represent a linker or a bond to the enzymatically generated C-terminal of FVIIa; and Z is a chemical moiety to be conjugated to the peptide.

13. A FVIIa derivative according to any one of embodiments 11 or 12, wherein P′ is the FVII polypeptide product of an enzymatic cleavage of the FVII polypeptides having the sequence of SEQ ID NO. 1 or SEQ ID NO 2.

14. The FVIIa derivative according to any one of embodiments 11-13, wherein the derivative is as produced by a method according to any one of embodiments 1-9.

15. The compounds

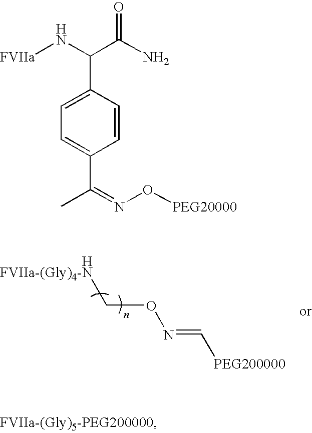

wherein n is ≧1, such as 1, such as 2, such as 3; wherein PEG20000 is a PEG moiety with a molecular weight of 20,000 Da.

16. A nucleic acid molecule encoding the FVII polypeptides according to embodiment 10.

17. A recombinant vector comprising the nucleic acid molecule according to embodiment 16.

18. A unicellular host organism containing a vector comprising the nucleic acid molecules according to embodiment 16.

19. The method according to any one of embodiments 1-9, wherein an enzyme selected from Sortase A and Sortase B has been applied.

21. The method according to embodiment 2, wherein Z is branched and contains one or more PEG with a molecular weight between about 10,000 Da and 40,000 Da.

1. A method of obtaining a derivate of FVIIa, P′—R—X, comprising the step of cleaving FVII or a FVII variant enzymatically in the presence of R′—X to conjugate —R—X at the enzymatically generated C-terminal of FVIIa: 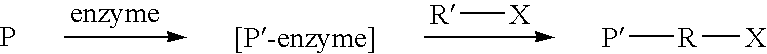
wherein P represents FVII or a FVII variant, P′ represents the product of the cleavage, R′—X represents the compound reacting with P′, X represents the group to be conjugated to P′ or X represents a functional group, R′ represents R comprising a nucleophilic group.

2. The method of claim 1, wherein the obtained product P′—R—X wherein X represents a functional group, is further reacted with a compound of the general formula Y-E-Z to obtain a product
P′—R-A-E-Z
wherein R represents a linker or a bond;
wherein P′ represents the FVII polypeptide product of the enzymatic enzymatically cleavage of FVII;
X represents a radical comprising a functional group capable of reacting with Y;
Y represents a radical comprising one or more functional groups which groups are capable of reacting with X;
E represents a linker or a bond;
A represents the moiety formed by the reaction between the functional groups comprised in X and Y; and
Z is the moiety to be conjugated to the peptide.

3. The method according to claim 1, wherein FVII is cleaved by FVIIa, FIXa, FSAP, Hepsin, or matriptase.

4. The method of claim 2, wherein X and Y are selected from carbonyl groups, keto and aldehyde groups, and amino derivatives, an amino acid, NH—NH₂, —NH—NH₂, —O—C(O)—NH—NH₂, —NH—C(O)—NH—NH₂, NH—C(S)—NH—NH₂, —NHC(O)—NH—NH—C(O)—NH—NH₂, NH—NH—C(O)—NH—NH₂, —NH—NH—C(S)—NH—NH₂, —NH—C(O)—C₆H₄—NH—NH₂, C(O)—NH—NH₂, —O—NH₂, —C(O)—O—NH₂, —NH—C(O)—O—NH₂ and —NH—C(S)—O—NH₂.

5. The method of claim 4, wherein Y is an amino acid, or a derivative of —NH—NH₂, —O—C(O)—NH—NH₂, NH—C(O)—NH—NH₂, —NH—C(S)—NH—NH₂, NHC(O)—NH—NH—C(O)—NH—NH₂, —NH—NH—C(O)—NH—NH₂, NH—NH—C(S)—NH—NH₂, —NH—C(O)—C₆H₄—NH—NH₂, —C(O)—NH—NH₂, —O—NH₂, —C(O)—O—NH₂, —NH—C(O)—O—NH₂ or —NH—C(S)—O—NH₂ and X is a keto- or an aldehyde-functionality.

6. The method of claim 1, wherein R′—X comprises one or more amino acids, wherein one or more of the amino acids are derivatized, and wherein one or more of the derivatized amino acids are optionally further derivatized, or the amino acid contains the group Z.

7. The method of claim 5, wherein R′—X is an α-amino acid derivative 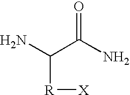
wherein R is a suitable linker and X is as defined in claim 1.

8. The method of claim 5, wherein R′—X is selected from the group consisting of G₍₁₋₅₎-PEG, G₍₁₋₅₎-lipid, G₍₁₋₄₎-NH—CH₂—CHO, and G₍₁₋₄₎-NH—(CH₂)ₙ—O—NH₂, wherein n is ≧2.

9. The method according to claim 1, wherein Z is PEG or C₅-C₂₄ fatty acid, aliphatic C₅-C₂₄diacid.

10. Isolated FVII polypeptides having the sequence of SEQ ID NO. 1 or SEQ ID NO 2.

11. A FVIIa derivative P′—R—X, wherein P′ represents a FVII polypeptide product of an enzymatic cleavage of FVII; X represents the group to be conjugated to P′ or X represents a functional group; R represent a linker or a bond to the enzymatically generated C-terminal of FVIIa.

12. A FVIIa derivative P′—R-A-E-Z, wherein P′ represents the FVII polypeptide product of an enzymatic cleavage of FVII; E represents a linker or a bond; A represents a chemical moiety; R represent a linker or a bond to the enzymatically generated C-terminal of FVIIa; and Z is a chemical moiety to be conjugated to the peptide.

13. A FVIIa derivative according to claim 11, wherein P′ is the FVII polypeptide product of an enzymatic cleavage of the FVII polypeptides having the sequence of SEQ ID NO. 1 or SEQ ID NO 2.

14. (canceled)

15. The compounds 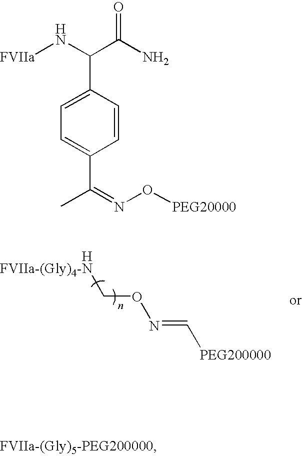
wherein n is ≧1; wherein PEG20000 is a PEG moiety with a molecular weight of 20,000 Da.

16. A nucleic acid molecule encoding the FVII polypeptides according to claim 10.

17. A recombinant vector comprising the nucleic acid molecule according to claim 16.

18. A unicellular host organism containing a vector comprising the nucleic acid molecules according to claim 16.

19. The method according to claim 1, wherein an enzyme selected from Sortase A and Sortase B has been applied.

20. The method according to claim 2, wherein Z is branched and contains one or more PEG with a molecular weight from about 10,000 Da to about 40,000 Da.

21. A FVIIa derivative according to claim 12, wherein P′ is the FVII polypeptide product of an enzymatic cleavage of the FVII polypeptides having the sequence of SEQ ID NO. 1 or SEQ ID NO 2.

22. A FVIIa derivative P′—R—X, wherein P′ represents a FVII polypeptide product of an enzymatic cleavage of FVII; X represents the group to be conjugated to P′ or X represents a functional group; R represent a linker or a bond to the enzymatically generated C-terminal of FVIIa, wherein the derivative is produced by the method of claim 1.

23. A FVIIa derivative P′—R-A-E-Z, wherein P′ represents the FVII polypeptide product of an enzymatic cleavage of FVII; E represents a linker or a bond; A represents a chemical moiety; R represent a linker or a bond to the enzymatically generated C-terminal of FVIIa; and Z is a chemical moiety to be conjugated to the peptide, wherein the derivative is produced by the method of claim 1.

24. The compounds of claim 15, wherein n is selected from 1, 2, or 3.

25. The FVIIa derivative according to claim 13, wherein the derivative is produced by a method according to claim 1

26. The FVIIa derivative according to claim 21, wherein the derivative is produced by a method according to claim 1.
